(19) United States
(12) Patent Application Publication
(10) Pub. No.: US 2018/0134795 A1
(43) Pub. Date: May 17, 2018

(54) CD123 SPECIFIC MULTI-CHAIN CHIMERIC ANTIGEN RECEPTOR

(71) Applicant: CELLECTIS, Paris (FR)

(72) Inventor: Roman GALETTO, Paris (FR)

(21) Appl. No.: 15/314,337

(22) PCT Filed: Jun. 17, 2015
(86) PCT No.: PCT/EP2015/063656

§ 371 Date: Nov. 28, 2016

(30) Foreign Application Priority Data
Jun. 17, 2014 (DK) PA201470361

(51) Int. Cl. C07K 16/28; C07K 14/735; C07K 14/705; C12N 15/85; A61K 35/17

(52) U.S. Cl. CPC C07K 16/2866; C07K 14/70535; C07K 2319/03; C12N 15/85; A61K 35/17; C07K 14/70517

(57) ABSTRACT

The present invention relates to a new generation of chimeric antigen receptors (CAR) referred to as multi-chain CARs, which are made specific to the antigen CD123. Such CARs aim to redirect immune cell specificity and reactivity toward malignant cells expressing the tumor antigen CD123. The alpha, beta and gamma polypeptides composing these CARs are designed to assemble in juxtamembrane position, which forms flexible architecture closer to natural receptors, that confers optimal signal transduction. The invention encompasses the polynucleotides, vectors encoding said multi-chain CAR and the isolated cells expressing them at their surface, in particularly for their use in immunotherapy. The invention opens the way to efficient adoptive immunotherapy strategies for treating cancer, especially leukemia.

### FIELD OF THE INVENTION

The present invention relates to a new generation of chimeric antigen receptors (CAR) referred to as multi-chain CARs, which are made specific to the antigen CD123. Such CARs aim to redirect immune cell specificity and reactivity toward malignant cells expressing the tumor antigen CD123. The alpha, beta and gamma polypeptides composing these CARs are designed to assemble in juxtamembrane position, which forms flexible architecture closer to natural receptors, that confers optimal signal transduction. The invention encompasses the polynucleotides, vectors encoding said multi-chain CAR and the isolated cells expressing them at their surface, in particularly for their use in immunotherapy. The invention opens the way to efficient adoptive immunotherapy strategies for treating cancer, especially acute myeloid leukemia (AML).

### BACKGROUND OF THE INVENTION

Adoptive immunotherapy, which involves the transfer of autologous antigen-specific T cells generated ex vivo, is a promising strategy to treat viral infections and cancer. The T cells used for adoptive immunotherapy can be generated either by expansion of antigen-specific T cells or redirection of T cells through genetic engineering (Park, Rosenberg et al. (2011) Treating Cancer with Genetically Engineered T Cells. Trends Biotechnol. 29(11): 550-557) Transfer of viral antigen specific T cells is a well-established procedure used for the treatment of transplant associated viral infections and rare viral-related malignancies. Similarly, isolation and transfer of tumor specific T cells has been shown to be successful in treating melanoma.

Novel specificities in T cells have been successfully generated through the genetic transfer of transgenic T cell receptors or chimeric antigen receptors (CARs) (Jena, Dotti et al. (2010) Redirecting T-cell specificity by introducing a tumor-specific chimeric antigen receptor. Blood. 116(7): 1035-1044). CARs are synthetic receptors consisting of a targeting moiety that is associated with one or more signaling domains to form a single-chain fusion molecule. However, this approach has so far proven efficiency only with respect to patients with acute lymphoblastic leukemia (ALL) by targeting malignant B cells bearing the antigen CD19 (Porter, D. L. et al. (2011) Chimeric Antigen Receptor-Modified T Cells in Chronic Lymphoid Leukemia. N. Engl. J. Med. 365:725-733).

Induction treatments for acute myeloid leukemia (AML) have remained largely unchanged for nearly 50 years and AML remains a disease of poor prognosis. Acute myeloid leukemia (AML) is a disease characterized by the rapid proliferation of immature myeloid cells in the bone marrow resulting in dysfunctional hematopoiesis. Although standard induction chemotherapy can induce complete remissions, many patients eventually relapse and succumb to the disease, calling for the development of novel therapeutics for AML.

Recent advances in the immunophenotyping of AML cells have revealed several AML associated cell surface antigens that may act as targets for future therapies. The interleukin 3 receptor alpha chain (IL-3Rα; CD123—NCBI reference: NP_001254642) has been identified as a potential immunotherapeutic target since it is over-expressed on AML tumor cells compared to normal hematopoietic stem cells. Additionally, two phase I trials for CD123-specific therapeutics have been completed with both drugs displaying good safety profiles (ClinicalTrials.gov ID: NCT00401739 and NCT00397579).

Unfortunately, these CD123 targeting drugs had limited efficacy suggesting that alternative and more potent therapies targeting CD123 are required to observe anti-leukemic activity.

In this regard, the CAR approach offered several advantages compared with monoclonal antibodies (mAbs), showing a more efficient biodistribution and improved synergism with the immune system through the release of cytokines. Moreover, a potential development of long-lasting cell-mediated immune responses could offer the possibility to durably control the disease overtime.

Drawbacks or side effects observed with the CAR approach such as hypercytokinemia were reported in patients undergoing such therapy after adoptive transfer of autologous cells. Thus, destruction of large tumor masses by autologous CD19-specific CAR-modified T cells has resulted in tumor lysis syndrome in patients with advanced CLL.

Further, graft versus host disease (GVHD) is a possibility after any allogeneic T-cell infusion. There is therefore a need for the development of new therapies targeting CD123 that would be more efficient and less toxic for the host.

In the context of developing therapeutic grade engineered immune cells that can target malignant or infected cells, the inventors have sought for improved CAR architectures, which would be closer to natural ones and likely to behave accordingly using any extracellular mono or multi-specific ligand binding domains. In WO2014039523, they described a new generation of CARs involving separate polypeptide sub-units according to the present invention, referred to as “multi-chain CARs”. According to this architecture, the signaling domains and co-stimulatory domains are located on different polypeptide chains. Such multi-chain CARs can be derived from FcϵRI, by replacing the high affinity IgE binding domain of FcϵRI alpha chain by an extracellular ligand-binding domain such as scFv, whereas the N and/or C-termini tails of FcϵRI beta and/or gamma chains are fused to signal transducing domains and co-stimulatory domains respectively. The extracellular ligand binding domain has the role of redirecting T-cell specificity towards cell targets, while the signal transducing domains activate the immune cell response. The fact that the different polypeptides derived from the alpha, beta and gamma polypeptides from FcϵRI are transmembrane polypeptides sitting in juxtamembrane position provides a more flexible architecture to CARs, improving specificity towards the targeted molecule and reducing background activation of immune cells.

The inventors have now designed multi-chain CAR bearing scFv extracellular domain binding CD123, which are particularly suited to target malignant cells bearing CD123 as a marker. This was achieved, whereas very few antibodies had been so far described to act efficiently against CD123 positive cells for treating or preventing leukemia, in particular AML.

For the purposes of the invention, inventors have now provided T cells expressing anti-CD123 multi chain CARS. Due to the design and architecture of these new anti-CD123 CARs and to the properties of the present engineered immune cells, the kinetic of action and activity of engineered immune cells is unexpectedly modified so that less tumor cells may escape and long term effect is observed with reduced GVHD and side effects (less toxic effects.

These original CARs specifically bind to and affect the survival of CD123 positive T cells, in particular to malignant CD123 positive cells developing during AML and selectively alter the viability of these malignant cells, with an expectation of displaying less toxic side effects including cytokine release.

### DESCRIPTION OF THE FIGURES

FIG. 1: Schematic representation of the native FcϵRI from which derivate the multi-chain CAR architecture according to the invention.

FIG. 2: General structure of the polycistronic construct encoding the CD123 multi-chain CAR according to the invention.

FIG. 3: Different architectures of the CD123 specific multi-chain CAR according to the invention. From left to right: polypeptide gamma (fused to ITAM of CD3zeta), polypeptide alpha (fused to ScFv), polypeptide beta (fused to co-stimulatory domain from either CD28 or 41BB). A and B: polypeptide beta is fused to co-stimulatory domain from 41BB, VL and VH fragments being in opposite orders. C and D: polypeptide beta is fused to co-stimulatory domain from CD28, VL and VH fragments being in opposite orders.

In FIG. 3C, and in FIG. 4, VL and VH fragments are in opposite order as compared to construction in FIG. 3D. In FIG. 3C and in FIG. 4, the VL fragment of the extracellular CD123 ligand binding domain is fused to a transmembrane polypeptide from the alpha chain of high-affinity IgE receptor (FcϵRI), more precisely to a peptide comprising a CD8 fragment and a fragment of the alpha chain of high-affinity IgE receptor (FcϵRI).

FIG. 4: Two architectures of the CD123 specific multi-chain CAR according to the invention (mc123-41BB and mc123-CD28) wherein the alpha fragment comprises a VL fragment is linked to a polypeptide comprising a CD8 fragment and to a VH fragment, and the beta chain comprises a co-stimulatory domain located in the C-terminus of the beta chain, said co-stimulatory domain is from 41BB (mc123-41BB) or from CD28 (mc123-CD28).

FIG. 5: Expression levels of mcCAR mc123-CD28, assessed 8 days after transduction at a MOI of 5.

CAR detection was performed using a recombinant fusion protein containing the extracellular domain of the human CD123 protein, fused to a mouse IgG1 derived Fc fragment. The CAR/CD123-Fc complex was detected using a PE-conjugated anti-Fc antibody and analyzed by flow cytometry. NTD stands for Non Transduced cells.

FIG. 6: Expression levels of mcCAR mc123-41BB, assessed 8 days after transduction at a MOI of 5. CAR detection was performed using a recombinant fusion protein containing the extracellular domain of the human CD123 protein, fused to a mouse IgG1 derived Fc fragment. The CAR/CD123-Fc complex was detected using a PE-conjugated anti-Fc antibody and analyzed by flow cytometry. NTD stands for Non Transduced cells.

FIG. 7: Degranulation activity of single chain (sc) CAR and mcCAR expressing T-cells when co-cultured for 6h with cells expressing different levels of CD123 (KG1a<MOLM13<RPMI8226), or with cells that do not express CD123 (Daudi).

The degranulation activity of T-cells cultured alone, in the same conditions that the co-cultures, is also shown; as the well as the positive control (cells activated with PMA/lonomycin). The degranulation activity was determined by flow cytometry, by measuring the % of CD107a+ cells (among CD8+ cells). The experiments were done in at least three independent donors.

FIG. 8: Specific cytolytic activity of mcCAR-T cells. T-cells were co-cultured with Daudi+KG1a, Daudi+MOLM13, or Daudi+RPMI-8226 cells for 4 hours. Cellular viability for each of the cell lines was determined at the end of the co-cultures and a specific cell lysis percentage was calculated for each condition. The results obtained show that both mcCAR targeting CD123 are expressed on T-cells and have comparable activity against CD123+ target cells.

TABLE 1
Exemplary sequences of the alpha polypeptide
component of CD123 multi-chain CAR

| Functional | | SEQ | Raw amino |
|---|---|---|---|
| domains | description | ID # | acid sequence |
| FcϵRI α-SP | signal peptide | SEQ ID | MAPAMESPTLLCVAL |
| | | NO. 1 | LFFAPDGVLA |
| CD8α hinge | hinge | SEQ ID | TTTPAPRPPTPAPTI |
| | | NO. 2 | ASQPLSLRPEACRPA |
| | | | AGGAVHTRGLDFACD |
| VH | | | See Table 5 |
| G4SX3Linker | Linker VH-VL | SEQ ID | GGGGSGGGGSGGGGS |
| | | NO. 3 | |
| VL | | | See Table 5 |
| FcϵRI | Fc Receptor for | SEQ ID | FFIPLLVVILFAVDT |
| α-TM-IC | IgE, alpha chain, | NO. 4 | GLFISTQQQVTFLLK |
| | transmembrane and | | IKRTRKGFRLLNPHP |
| | intracellular | | KPNPKNN |
| | domain | | |

TABLE 2
Exemplary sequences of the beta polypeptide
component of CD123 multi-chain CAR

| Functional | | SEQ | Raw amino |
|---|---|---|---|
| domains | description | ID # | acid sequence |
| FcϵR1β- | Fc Receptor | SEQ ID | MDTESNRRANLALPQEPSSVP |
| ΔITAM | for IgE, | NO. 5 | AFEVLEISPQEVSSGRLLKSA |
| | beta chain, | | SSPPLHTWLTVLKKEQEFLGV |
| | without | | TQILTAMICLCFGTVVCSVLD |
| | ITAM | | ISHIEGDIFSSFKAGYPFWGA |
| | | | IFFSISGMLSIISERRNATYL |
| | | | VRGSLGANTASSIAGGTGITI |
| | | | LIINLKKSLAYIHIHSCQKFF |
| | | | ETKCFMASFSTEIVVMMLFLT |
| | | | ILGLGSAVSLTICGAGEELKG |
| | | | NKVPE |
| 41BB-IC | 41BB co- | SEQ ID | KRGRKKLLYIFKQPFMRPVQT |
| | stimulatory | NO. 6 | TQEEDGCSCRFPEEEEGGCEL |
| | domain | | |
| CD28-IC | CD28 co- | SEQ ID | RSKRSRGGHSDYMNMTPRRPG |
| | stimulatory | NO. 7 | PTRKHYQPYAPPRDFAAYRS |
| | domain | | |

TABLE 3
Exemplary sequences of the gamma polypeptide
component of CD123 multi-chain CAR

| Functional | | SEQ | Raw amino |
|---|---|---|---|
| domains | description | ID # | acid sequence |
| FcϵRI γ-SP | signal | SEQ ID | MIPAVVLLLLLLVEQ |
| | peptide | NO. 8 | AAA |
| FcϵRI | Fc Receptor | SEQ ID | LGEPQLCYILDAILF |
| γ-ΔITAM | for IgE, | NO. 9 | LYGIVLTLLYCRLKI |
| | gamma chain, | | QVRKAAITSYEKS |
| | without ITAM | | |
| CD3ζ-IC | CD3zeta | SEQ ID | RVKFSRSADAPAYQQ |
| | intracellular | NO. 10 | ELNLGRREEYDVLDK |
| | domain | | RRGGQNQLYNRDPEM |
| | comprising | | GGKPRRKNPQEGLYN |
| | ITAM | | ELQKDKMAEAYSEIG |
| | | | MKGERRRGKGHDGLY |
| | | | QGLSTATKDTYDALH |
| | | | MQALPPR |

TABLE 4
skip peptides linking the polypeptides
forming the multi-subunit CAR

| Functional | | SEQ | Raw amino |
|---|---|---|---|
| domains | description | ID # | acid sequence |
| GSG-P2A | GSG-P2A ribosomal | SEQ ID | GSGATNFSLLKQA |
| | skip peptide | NO. 11 | GDVEENPGP |
| GSG-T2A | GSG-T2A ribosomal | SEQ ID | GSGEGRGSLLTCG |
| | skip peptide | NO. 12 | DVEENPGP |

TABLE 5
Sequence of exemplary CD123 binding regions

| CD123 ScFv sequences | SEQ ID # | Raw amino acid sequence |
|---|---|---|
| anti-CD123 7G3 heavy | SEQ ID | MGWSWIFLFLVSGTGGVLSEVQLQQSGPEL |
| chain variable region | NO. 13 | VKPGASVKMSCKASGYTFTDYYMKWVKQSH |
| | | GKSLEWIGDIIPSNGATFYNQKFKGKATLTVD |
| | | RSSSTAYMHLNSLTSEDSAVYYCTRSHLLRAS |
| | | WFAYWGQGTLVTVSAAS |
| anti-CD123 7G3 light | SEQ ID | MESQTQVLMSLLFWVSGTCGDFVMTQSPSS |
| chain variable region | NO. 14 | LTVTAGEKVTMSCKSSQSLLNSGNQKNYLTW |
| | | YLQKPGQPPKLLIYWASTRESGVPDRFTGSGS |
| | | GTDFTLTISSVQAEDLAVYYCQNDYSYPYTFG |
| | | GGTKLEIKR |
| anti-CD123 Old4 heavy | SEQ ID | WTWRFLFVVAAATGVQSQVQLLQSGAEVKK |
| chain variable region | NO. 15 | PGSSVKVSCKASGGTFSTYAISWVRQAPGQG |
| | | LEWMGGIIPIFGIVNYAQKFQGRVTITADEST |
| | | STAYMELSSLRSEDTAVYYCARGGGSGPDVL |
| | | DIWGQGTMVTVSSAST |
| anti-CD123 Old4 light | SEQ ID | MDMRVPAQLLGLLLLWLPGARCVIWMTQS |
| chain variable region | NO. 16 | PSLLSASTGDRVTISCRMSQGIRSYLAWYQQK |
| | | PGKAPELLIYAASTLQSGVPSRFSGSGSGTDFT |
| | | LTISSLQSEDFATYYCQQYYSFPYTFGQGTKLE |
| | | IKRTV |
| anti-CD123 26292 heavy | SEQ ID | QVQLQQPGAELVRPGASVKLSCKASGYTFTS |
| chain variable region | NO. 17 | YWMNWVKQRPDQGLEWIGRIDPYDSETHY |
| | | NQKFKDKAILTVDKSSSTAYMQLSSLTSEDSA |
| | | VYYCARGNWDDYWGQGTTLTVSS |
| anti-CD123 26292 light | SEQ ID | DVQITQSPSYLAASPGETITINCRASKSISKDLA |
| chain variable region | NO. 18 | WYQEKPGKTNKLLIYSGSTLQSGIPSRFSGSGS |
| | | GTDFTLTISSLEPEDFAMYYCQQHNKYPYTFG |
| | | GGTKLEIK |
| anti-CD123 32716 heavy | SEQ ID | QIQLVQSGPELKKPGETVKISCKASGYIFTNYG |
| chain variable region | NO. 19 | MNWVKQAPGKSFKWMGWINTYTGESTYSA |
| | | DFKGRFAFSLETSASTAYLHINDLKNEDTATYF |
| | | CARSGGYDPMDYWGQGTSVTVSS |
| anti-CD123 32716 light | SEQ ID | DIVLTQSPASLAVSLGQRATISCRASESVDNY |
| chain variable region | NO. 20 | GNTFMHWYQQKPGQPPKLLIYRASNLESGIP |
| | | ARFSGSGSRTDFTLTINPVEADDVATYYCQQS |
| | | NEDPPTFGAGTKLELK |
| anti-CD123 Klon43 heavy | SEQ ID | EVKLVESGGGLVQPGGSLSLSCAASGFTFTDYYMS |
| chain variable region | NO. 21 | WVRQPPGKALEWLALIRSKADGYTTEYSASVKGR |
| | | FTLSRDDSQSILYLQMNALRPEDSATYYCARDAAY |
| | | YSYYSPEGAMDYWGQGTSVTVSS |
| anti-CD123 Klon43 light | SEQ ID | MADYKDIVMTQSHKFMSTSVGDRVNITCKASQN |
| chain variable region | NO. 22 | VDSAVAWYQQKPGQSPKALIYSASYRYSGVPDRF |
| | | TGRGSGTDFTLTISSVQAEDLAVYYCQQYYSTPWT |
| | | FGGGTKLEIKR |
| anti-CD123 12F1 heavy | SEQ ID | VQLQESGPGLVKPSQSLSLTCSVTDYSITSGYY |
| chain variable region | NO. 23 | WNWIRQFPGNKLEWMGYISYDGSNNYNPS |
| | | LKNRISITRDTSKNQFFLKLSSVTTEDTATYYCS |
| | | RGEGFYFDSWGQGTTLTVSSARS |
| anti-CD123 12F1 light | SEQ ID | DIMMSQSPSSLAVSVGEKFTMTCKSSQSLFF |
| chain variable region | NO. 24 | GSTQKNYLAWYQQKPGQSPKLLIYWASTRES |
| | | GVPDRFTGSGSGTDFTLAISSVMPEDLAVYYC |
| | | QQYYNYPWTFGGGTKLEIK |

TABLE 6
Exemplary Polypeptides forming anti-CD123 multi-chain CAR

| | Precursor CD123 multi-chain CAR polypeptide structure | | | | | | | | | | | | |
|---|---|---|---|---|---|---|---|---|---|---|---|---|---|
| | | | | | | | | | | | | Beta polypeptide | |
| | | | | | | | | | | | | | Co- |
| Multi chain | Gamma polypeptide | | | | Alpha polypeptide | | | | | | | | stimu- |
| CAR | FcεRI | FcεRI γ- | | | FcεRI α- | CD8α | | G4SX3 | | FcεRI α- | | FcεR1β- | lalion. |
| Designation | γ-SP | ΔITAM | CD3ζ-IC | P2A | SP | hinge | VH | Linker | VL | TM-IC | T2A | ΔITAM | domain |
| anti-CD123 | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
| 7G3 | NO. 8 | NO. 9 | NO. 10 | NO. 11 | NO. 1 | NO. 2 | NO. 13 | NO. 3 | NO. 14 | NO. 4 | NO. 12 | NO. 5 | NO. 6 |
| (41BB) | | | | | | | | | | | | | |
| anti-CD123 | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
| 7G3 | NO. 8 | NO. 9 | NO. 10 | NO. 11 | NO. l | NO. 2 | NO. 13 | NO. 3 | NO. 14 | NO. 4 | NO. 12 | NO. 5 | NO. 7 |
| (CD28) | | | | | | | | | | | | | |
| anti-CD123 | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
| Old4 | NO. 8 | NO. 9 | NO. 10 | NO. 11 | NO. l | NO. 2 | NO. 15 | NO. 3 | NO. 16 | NO. 4 | NO. 12 | NO. 5 | NO. 6 |
| (41BB) | | | | | | | | | | | | | |
| anti-CD123 | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
| Old4 | NO. 8 | NO. 9 | NO. 10 | NO. 11 | NO. l | NO. 2 | NO. 15 | NO. 3 | NO. 16 | NO. 4 | NO. 12 | NO. 5 | NO. 7 |
| (CD28) | | | | | | | | | | | | | |
| anti-CD123 | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
| 26292 | NO. 8 | NO. 9 | NO. 10 | NO. 11 | NO. l | NO. 2 | NO. 17 | NO. 3 | NO. 18 | NO. 4 | NO. 12 | NO. 5 | NO. 6 |
| (41BB) | | | | | | | | | | | | | |
| anti-CD123 | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
| 26292 | NO. 8 | NO. 9 | NO. 10 | NO. 11 | NO. l | NO. 2 | NO. 17 | NO. 3 | NO. 18 | NO. 4 | NO. 12 | NO. 5 | NO. 7 |
| (CD28) | | | | | | | | | | | | | |
| anti-CD123 | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
| 32716 | NO. 8 | NO. 9 | NO. 10 | NO. 11 | NO. l | NO. 2 | NO. 19 | NO. 3 | NO. 20 | NO. 4 | NO. 12 | NO. 5 | NO. 6 |
| (41BB) | | | | | | | | | | | | | |
| anti-CD123 | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
| 32716 | NO. 8 | NO. 9 | NO. 10 | NO. 11 | NO. 1 | NO. 2 | NO. 19 | NO. 3 | NO. 20 | NO. 4 | NO. 12 | NO. 5 | NO. 7 |
| (CD28) | | | | | | | | | | | | | |
| anti-CD123 | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
| Klon43 | NO. 8 | NO. 9 | NO. 10 | NO. 11 | NO. l | NO. 2 | NO. 21 | NO. 3 | NO. 22 | NO. 4 | NO. 12 | NO. 5 | NO. 6 |
| (41BB) | | | | | | | | | | | | | |
| anti-CD123 | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
| Klon43 | NO. 8 | NO. 9 | NO. 10 | NO. 11 | NO. l | NO. 2 | NO. 21 | NO. 3 | NO. 22 | NO. 4 | NO. 12 | NO. 5 | NO. 7 |
| (CD28) | | | | | | | | | | | | | |
| anti-CD123 | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
| 12F1 | NO. 8 | NO. 9 | NO. 10 | NO. 11 | NO. l | NO. 2 | NO. 23 | NO. 3 | NO. 24 | NO. 4 | NO. 12 | NO. 5 | NO. 6 |
| (41BB) | | | | | | | | | | | | | |
| anti-CD123 | SEQ ID | SEQ ID | SEQ ID | SEQ ID | SEQ ID | SEQ ID | SEQ ID | SEQ ID | SEQ ID | SEQ ID | SEQ ID | SEQ ID | SEQ ID |
| 12F1 | NO. 8 | NO. 9 | NO. 10 | NO. 11 | NO. l | NO. 2 | NO. 23 | NO. 3 | NO. 24 | NO. 4 | NO. 12 | NO. 5 | NO. 7 |
| (CD28) | | | | | | | | | | | | | |

Unless specifically defined herein, all technical and scientific terms used have the same meaning as commonly understood by a skilled artisan in the fields of gene therapy, biochemistry, genetics, and molecular biology. Document PCT/EP2015/055848 is incorporated herein by reference in its entirety.

All methods and materials similar or equivalent to those described herein can be used in the practice or testing of the present invention, with suitable methods and materials being described herein and in PCT/EP2015/055848. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols.154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

In particular, the present invention provides a CD123 specific multi-chain Chimeric Antigen Receptor (mc CAR) comprising:
A transmembrane polypeptide from the alpha chain of high-affinity IgE receptor (FcϵRI) fused to an extracellular CD123 ligand binding domain.

The present invention provides a CD123 specific multi-chain Chimeric Antigen Receptor (mc CAR) as above further comprising:
A second transmembrane polypeptide from the gamma or beta chain of FcϵRI fused to a signal transducing domain;

The present invention provides a CD123 specific multi-chain Chimeric Antigen Receptor (mc CAR) according as above, further comprising:
A third transmembrane polypeptide from the gamma or beta chain of FcϵRI comprising a co-stimulatory domain.

The present invention provides a CD123 specific multi-chain Chimeric Antigen Receptor according to any one of the above, wherein said CD123 ligand binding domain fused to said alpha chain of FcϵRI is a single-chain variable fragment (scFv) comprising heavy (V_{H}) and light (V_{L}) chains conferring specificity to CD123.

The present invention provides a CD123 specific multi-chain Chimeric Antigen Receptor as above, wherein said V_{H} comprises a polypeptide sequence displaying at least 90% identity to one selected from SEQ ID NO. 13, 15, 17, 19, 21 and 23.

The present invention provides a CD123 specific multi-chain Chimeric Antigen Receptor as above, wherein said V_{L} comprises a polypeptide displaying at least 90% identity to one selected from SEQ ID NO. 14, 16, 18, 20, 22 and 24.

The present invention provides a CD123 specific multi-chain Chimeric Antigen Receptor as above, wherein said alpha chain of FcϵRI is fused to said extracellular ligand-binding domain by a hinge from CD8α, IgG1 or FcRIIIα proteins.

The present invention provides a CD123 specific multi-chain Chimeric Antigen Receptor as above, wherein said hinge comprises a polypeptide sequence displaying at least 90% identity to SEQ ID NO.2.

The present invention provides a CD123 specific multi-chain Chimeric Antigen Receptor according to any one of the above, wherein said signal transducing domain fused to the gamma or beta chain of FcϵRI is from the TCR zeta chain, the FCϵRβ chain, the FcϵRIγ chain, or includes an immunoreceptor tyrosine-based activation motif (ITAM).

The present invention provides a CD123 specific multi-chain Chimeric Antigen Receptor as above, wherein said signal transducing domain is from CD3zeta.

The present invention provides a CD123 specific multi-chain Chimeric Antigen Receptor as above, wherein said signal transducing domain comprises a polypeptide sequence displaying at least 90% identity to SEQ ID NO.10.

The present invention provides a CD123 specific multi-chain Chimeric Antigen

Receptor as above, wherein said second or third polypeptide comprises a co-stimulatory domain from the cytoplasmic domain of a costimulatory molecule selected from CD27, CD28, 4-1BB, OX40, CD30, CD40, PD-1, ICOS, lymphocyte function-associated antigen-1 (LFA-1), CD2, CD7, CD8, LIGHT, NKG2C, B7-H3, a ligand that specifically binds with CD83, and any combination thereof.

The present invention provides a CD123 specific multi-chain Chimeric Antigen Receptor as above, wherein said co-stimulatory domain is from 4-1BB and comprises a polypeptide sequence displaying at least 90% identity to SEQ ID NO.6.

The present invention provides a CD123 specific multi-chain Chimeric Antigen Receptor as above, wherein said co-stimulatory domain is from CD28 and comprises a polypeptide sequence displaying at least 90% identity to SEQ ID NO.7.

The present invention provides a polypeptide encoding a CD123 specific multi-chain Chimeric Antigen Receptor as above, comprising a polypeptide sequence displaying at least 80% identity to the full amino acid sequence of anti-CD123 7G3, anti-CD123 Old4, anti-CD123 26292, anti-CD123 32716, anti-CD123 Klon43, anti-CD123 12F1 as referred to in Table 6.

The present invention provides a polynucleotide comprising a nucleic acid sequence encoding a CD123 specific multi-chain Chimeric Antigen Receptor as above.

The present invention provides a vector comprising a polynucleotide as above,The present invention provides a method of engineering an immune cell comprising:
(a) Providing an immune cell;
(b) Expressing at the surface of said cells at least one multi-chain Chimeric Antigen Receptor as above.

The present invention provides a method of engineering an immune cell as above comprising:
(a) Providing an immune cell;
(b) Introducing into said cell at least one polynucleotide encoding polypeptides composing at least one multi-chain Chimeric Antigen Receptor as above;
(c) Expressing said polynucleotides into said cell.

The present invention also provides with a method of engineering an immune cell as above comprising the following steps of:
(a) Providing an immune cell;
(b) Expressing at the surface of said cell a population of multi-chain Chimeric

Antigen Receptors as above each one comprising different extracellular ligand-binding domains.

The present invention provides a method of engineering an immune cell as above comprising:
(a) Providing an immune cell;
(b) Introducing into said cell at least one polynucleotide encoding polypeptides composing a population of multi-chain Chimeric Antigen Receptors as above each one comprising different extracellular ligand binding domains.
(c) Expressing said polynucleotides into said cell.

The present invention provides an isolated immune cell obtainable from the method as above.

The present invention provides an isolated immune cell comprising at least one multi-chain Chimeric Antigen Receptor as above.

The present invention provides an isolated cell as above derived from, NK cells, inflammatory T-lymphocytes, cytotoxic T-lymphocytes, regulatory T-lymphocytes or helper T-lymphocytes.

The present invention provides an isolated immune cell as above for its use as a medicament.

The present invention further provides a method for treating a patient in need thereof comprising:
a) Providing a immune cell obtainable by a method as above;
b) Administrating said T-cells to said patient,

The present invention provides a method for treating a patient as above wherein said immune cells are recovered from donors or patients.

CD123 Multi chain CARs

The present invention provides a CD123 specific multi-chain Chimeric Antigen Receptor (mc CAR) comprising:
A transmembrane polypeptide from the alpha chain of high-affinity IgE receptor (FcϵRI) fused to an extracellular CD123 ligand binding domain.

The present invention provides a CD123 specific multi-chain Chimeric Antigen Receptor (mc CAR) as above further comprising:
A second transmembrane polypeptide from the gamma or beta chain of FcϵRI fused to a signal transducing domain.

The present invention provides a CD123 specific multi-chain Chimeric Antigen Receptor (mc CAR) as above comprising:
A third transmembrane polypeptide from the gamma or beta chain of FcϵRI comprising a co-stimulatory domain.

A CD123 specific multi-chain Chimeric Antigen Receptor (CD123 mc CAR or anti-CD123 mc CAR) means a multi-chain Chimeric Antigen Receptor that specifically binds to CD123, preferably a CD123 specific multi-chain Chimeric Antigen means a multi-chain Chimeric Antigen Receptor that specifically binds to CD123 and affects the survival of a CD123 expressing cell, in particular a CD123 expressing cancer cell.

In a preferred embodiment, the present invention provides a CD123 specific multi-chain Chimeric Antigen Receptor (CD123 mc CAR) comprising:
A transmembrane polypeptide from the alpha chain of high-affinity IgE receptor (FcϵRI) fused to an extracellular CD123 ligand binding domain,
a second transmembrane polypeptide from the gamma or beta chain of FcϵRI fused to a signal transducing domain; and
a third transmembrane polypeptide from the gamma or beta chain of FcϵRI comprising a co-stimulatory domain.

In one embodiment, the present invention provides a CD123 specific multi-chain Chimeric Antigen Receptor (mc CAR) comprising:
a transmembrane polypeptide from the alpha chain of high-affinity IgE receptor (FcϵRI) fused to an extracellular CD123 ligand binding domain,
   a second transmembrane polypeptide from the gamma or beta chain of FcϵRI fused to a signal transducing domain; and
   a third transmembrane polypeptide from the gamma or beta chain of FcϵRI comprising a co-stimulatory domain.
      wherein said extra cellular ligand binding-domain comprising a VH and a VL from a monoclonal anti-CD123 antibody comprises the following CDR sequences:
   GFTFTDYY (SEQ ID NO. 26), RSKADGYTT (SEQ ID NO. 27), ARDAAYYSYYSPEGAMDY (SEQ ID NO. 28), and QNVDSA (SEQ ID NO. 29), SAS (SEQ ID NO. 30), QQYYSTPWT (SEQ ID NO. 31).

In one embodiment, the present invention provides a CD123 specific multi-chain Chimeric Antigen Receptor (CD123 mc CAR) comprising:
a first transmembrane polypeptide from the alpha chain of high-affinity IgE receptor (FcϵRI) fused to an extracellular CD123 ligand binding domain,
a second transmembrane polypeptide from the gamma or beta chain of FcϵRI fused to a signal transducing domain; and
a third transmembrane polypeptide from the gamma or beta chain of FcϵRI comprising a co-stimulatory domain.
   wherein said extra cellular ligand binding-domain comprising a VH and a VL from a monoclonal anti-CD123 antibody comprises the following CDR sequences:
GFTFTDYY (SEQ ID NO. 44), RSKADGYTT (SEQ ID NO. 45), ARDAAYYSYYSPEGAMDY (SEQ ID NO. 46), and QNVDSA (SEQ ID NO. 47), SAS (SEQ ID NO. 48), QQYYSTPWT (SEQ ID NO. 49), and a hinge between VH and VL (alpha chain),
wherein said signal transducing domain (or cytoplasmic transmembrane domain) comprises a CD3 zeta signaling domain (gamma chain), and
wherein said co-stimulatory domain comprises a co-stimulatory transmembrane domain from 4-1BB or CD28 (beta chain).

In an even more preferred embodiment, the present invention provides a CD123 specific multi-chain Chimeric Antigen Receptor (CD123 mc CAR) as any of the above embodiments, comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or a CD123 specific multi-chain Chimeric Antigen Receptor (mc CAR) comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

In this embodiment, the present invention provides a CD123 specific multi-chain Chimeric Antigen Receptor (CD123 mc CAR), wherein the polypeptide sequences has at least 90% identity with the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or wherein the polypeptide sequences has at least 90% identity with the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

The present invention provides a CD123 specific multi-chain Chimeric Antigen Receptor (CD123 mc CAR), wherein the polypeptide sequences has at least 95% identity with the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or wherein the polypeptide sequences has at least 95% identity with the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

The present invention provides a CD123 specific multi-chain Chimeric Antigen Receptor (CD123 mc CAR), wherein the polypeptide sequences has at least 98% identity with the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or wherein the polypeptide sequences has at least 98% identity with the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

The present invention provides a CD123 specific multi-chain Chimeric Antigen Receptor (CD123mc CAR), wherein the polypeptide sequences has at least 99% identity with the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or wherein the polypeptide sequences has at least 99% identity with the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

In all the above embodiments, said CD123 specific multi-chain Chimeric Antigen Receptor (CD123 mc CAR), retains, continuously or temporarily, their properties of binding to CD123 expressing cells and/or to affect the survival of said CD123 expressing cancer cells.

The present invention provides a CD123 specific multi-chain Chimeric Antigen Receptor as above, wherein said CD123 ligand binding domain fused to said alpha chain of

FcϵRI is a single-chain variable fragment (scFv) comprising a heavy (V_{H}) and a light (V_{L}) chain conferring specificity to CD123, preferably from antibody “Kion 43”.

In a preferred embodiment, the present invention provides a CD123 specific multi-chain Chimeric Antigen Receptor as above, wherein said CD123 ligand binding domain fused to said alpha chain of FcϵRI is a single-chain variable fragment (scFv) derived (or has at least 90% from 99% identity with) from antibody “Kion 43” comprising a heavy (V_{H}) and a light (V_{L}) chains conferring specificity to CD123 or is derived from an humanized “Kion 43” antibody comprising a heavy (V_{H}) and a light (V_{L}) chains conferring specificity to human CD123.

In a more preferred embodiment, the present invention provides a CD123 specific multi-chain Chimeric Antigen Receptor as above, wherein said CD123 ligand binding domain fused to said alpha chain of FcϵRI is a single-chain variable fragment (scFv) derived from antibody “Kion 43” having between from 90% to 100% identity with SEQ ID NO. 21 and SEQ ID NO. 22.

The present invention provides a CD123 specific multi-chain Chimeric Antigen Receptor as above, wherein said V_{H} comprises a polypeptide sequence having at least 100% to at least 90% identity with one of the polypeptide sequences selected from SEQ ID NO. 13, SEQ ID NO. 15, SEQ ID NO. 17, SEQ ID NO. 19, SEQ ID NO. 21 and SEQ ID NO. 23.

The present invention provides a CD123 specific multi-chain Chimeric Antigen Receptor as above wherein said V_{L} comprises a polypeptide having at least 100% to at least 90% identity with one of the polypeptide sequences selected from SEQ ID NO. 14, SEQ ID NO. 16, SEQ ID NO. 18, SEQ ID NO. 20, SEQ ID NO. 22 and SEQ ID NO. 24.

The present invention provides a CD123 specific multi-chain Chimeric Antigen Receptor as above, wherein said alpha chain of FcϵRI is fused to said extracellular ligand-binding domain by a hinge from CD8α, IgG1 or FcRIIIα proteins.

The present invention provides a CD123 specific multi-chain Chimeric Antigen Receptor as above, wherein said hinge comprises a polypeptide sequence displaying at least 90% identity with SEQ ID NO.2. In one preferred embodiment, said hinge comprises a polypeptide of SEQ ID NO.2.

The present invention provides a CD123 specific multi-chain Chimeric Antigen Receptor according to any one of the above embodiments, wherein said signal transducing domain fused to the gamma or beta chain of FcϵRI is from the TCR zeta chain, the FCϵRβ chain, the FcϵRIγ chain, or includes an immunoreceptor tyrosine-based activation motif (ITAM).

The present invention provides a CD123 specific multi-chain Chimeric Antigen Receptor according to any one of the above embodiments, wherein said signal transducing domain fused to the gamma or beta chain of FcϵRI is from the TCR zeta chain.

The present invention provides a CD123 specific multi-chain Chimeric Antigen Receptor according to any one of the above embodiments, wherein said signal transducing domain fused to the gamma or beta chain of FcϵRI is from the FCϵRβ chain.

The present invention provides a CD123 specific multi-chain Chimeric Antigen Receptor according to any one of the above embodiments, wherein said signal transducing domain fused to the gamma or beta chain of FcϵRI is from the FcϵRIγ chain.

The present invention provides a CD123 specific multi-chain Chimeric Antigen Receptor according to any one of the above embodiments, wherein said signal transducing domain fused to the gamma or beta chain of FcϵRI comprises an immunoreceptor tyrosine-based activation motif (ITAM).

The present invention provides a CD123 specific multi-chain Chimeric Antigen Receptor as above, wherein said signal transducing domain is from CD3zeta, preferably the present invention provides a CD123 specific multi-chain Chimeric Antigen Receptor as above, wherein said signal transducing domain comprises a polypeptide sequence displaying at least 90% identity to SEQ ID NO.10. In one embodiment, said signal transducing domain comprises a polypeptide sequence of SEQ ID NO.10.

The present invention is related to a CD123 specific multi-chain Chimeric Antigen Receptor as any of the above embodiment, wherein said second or third polypeptide comprises a co-stimulatory domain from the cytoplasmic domain of a costimulatory molecule selected from CD27, CD28, 4-1BB, 0X40, CD30, CD40, PD-1, ICOS, lymphocyte function-associated antigen-1 (LFA-1), CD2, CD7, CD8, LIGHT, NKG2C, B7-H3, a ligand that specifically binds with CD83, and any combination thereof.

In a preferred embodiment, the present invention is related to a CD123 specific multi-chain Chimeric Antigen Receptor as any of the above embodiment, wherein said second or third polypeptide comprises a co-stimulatory domain from the cytoplasmic domain of a costimulatory molecule from CD28.

In a preferred embodiment, the present invention is related to a CD123 specific multi-chain Chimeric Antigen Receptor as any of the above embodiment, wherein said second or third polypeptide comprises a co-stimulatory domain from the cytoplasmic domain of a costimulatory molecule from 4-1BB.

The present invention provides a CD123 specific multi-chain Chimeric Antigen Receptor as above, wherein said co-stimulatory domain is from 4-1BB and comprises a polypeptide sequence displaying at least 90% identity with SEQ ID NO.6. In one embodiment, said co-stimulatory domain is from 4-1BB and comprises a polypeptide sequence of SEQ ID NO.6.

The present invention provides a CD123 specific multi-chain Chimeric Antigen Receptor as above, wherein said co-stimulatory domain is from CD28 and comprises a polypeptide sequence displaying at least 90% identity to SEQ ID NO.7. In one embodiment, said co-stimulatory domain is from CD28 and comprises a polypeptide sequence having SEQ ID NO.7.

The present invention provides a polypeptide encoding a CD123 specific multi-chain Chimeric Antigen Receptor as above, comprising a polypeptide sequence displaying at least 80% identity to the full amino acid sequence of anti-CD123 7G3, anti-CD123 Old4, anti-CD123 26292, anti-CD123 32716, anti-CD123 Klon43, anti-CD123 12F1 as referred to in Table 6.

In a preferred embodiment, the present invention provides a polypeptide encoding a CD123 specific multi-chain Chimeric Antigen Receptor as disclosed above, comprising a polypeptide sequence displaying at least 80% identity to the full amino acid sequence of anti-CD123 Klon43 VH and VL, preferably at least 80% identity with SEQ ID NO. 21 and SEQ ID NO. 22.

In a more preferred embodiment, the present invention provides a polypeptide encoding a CD123 specific multi-chain Chimeric Antigen Receptor as disclosed above, comprising a polypeptide sequence displaying at least 90% identity to the full amino acid sequence of anti-CD123 Klon43 VH and VL, preferably at least 90% identity with SEQ ID NO. 21 and SEQ ID NO. 22.

In another more preferred embodiment, the present invention provides a polypeptide encoding a CD123 specific multi-chain Chimeric Antigen Receptor as disclosed above, comprising a polypeptide sequence displaying 100% identity to the full amino acid sequence of anti-CD123 Klon43 VH and VL preferably with SEQ ID NO. 21 and SEQ ID NO. 22.

The present invention preferably provides a CD123 specific multi-chain Chimeric Antigen Receptor (mc CAR) comprising:
a first transmembrane polypeptide from the alpha chain of high-affinity IgE receptor (FcϵRI) fused to an extracellular ligand binding domain specifically binding to CD123 comprising a single-chain variable fragment (scFv) comprising a heavy (V_{H}) and a light (V_{L}) chain conferring specificity to CD123,
a second transmembrane polypeptide from the gamma or beta chain of FcϵRI fused to a signal transducing domain; and
a third transmembrane polypeptide from the gamma or beta chain of FcϵRI comprising a co-stimulatory domain.

In one embodiment, said extra cellular ligand binding-domain comprising a VH and a VL from a monoclonal anti-CD123 antibody, comprises the following CDR sequences:
GFTFTDYY (SEQ ID NO. 26), RSKADGYTT (SEQ ID NO. 27), ARDAAYYSYYSPEGAMDY (SEQ ID NO. 28), and QNVDSA (SEQ ID NO. 29), SAS (SEQ ID NO. 30), QQYYSTPWT (SEQ ID NO. 31).

In a more preferred embodiment, the present invention provides a CD123 specific multi-chain Chimeric Antigen Receptor (mc CAR) comprising the following CDR sequences:
GFTFTDYY (SEQ ID NO. 44), RSKADGYTT (SEQ ID NO. 45), ARDAAYYSYYSPEGAMDY (SEQ ID NO. 46), and QNVDSA (SEQ ID NO. 47), SAS (SEQ ID NO. 48), QQYYSTPWT (SEQ ID NO. 49), and, further comprising the following peptide sequences: SEQ ID NO.8, SEQ ID NO.9, SEQ ID NO.10, SEQ ID NO.11, SEQ ID NO.1, SEQ ID NO.2, SEQ ID NO.3, SEQ ID NO.4, SEQ ID NO.12, SEQ ID NO.5, and SEQ ID NO.6 or the following peptide sequences: SEQ ID NO.8, SEQ ID NO.9, SEQ ID NO.10, SEQ ID NO.11, SEQ ID NO.1, SEQ ID NO.2, SEQ ID NO.3, SEQ ID NO.4, SEQ ID NO.12, SEQ ID NO.5, and SEQ ID NO.7.

The present invention provides a CD123 specific multi-chain Chimeric Antigen Receptor as above, wherein said alpha chain of FcϵRI is fused to said extracellular ligand-binding domain by a hinge from CD8α, IgG1 or FcRIIIα proteins, preferably said hinge comprises a polypeptide of SEQ ID NO.2.

The present invention provides a CD123 specific multi-chain Chimeric Antigen Receptor according to any one of the above embodiments, wherein said signal transducing domain fused to the gamma or beta chain of FcϵRI is from the TCR zeta chain, the FCϵRβ chain, the FcϵRIγ chain, or includes an immunoreceptor tyrosine-based activation motif (ITAM), preferably said signal transducing domain is from CD3zeta, more preferably comprising a polypeptide sequence of SEQ ID NO.10.

The present invention is related to CD123 specific multi-chain Chimeric Antigen Receptor as any of the above embodiment, wherein said second or third transmembrane polypeptide comprises a co-stimulatory domain from the cytoplasmic domain of a costimulatory molecule selected from CD27, CD28, 4-1BB, 0X40, CD30, CD40, PD-1, ICOS, lymphocyte function-associated antigen-1 (LFA-1), CD2, CD7, CD8, LIGHT, NKG2C, B7-H3, a ligand that specifically binds with CD83, and any combination thereof.

The present invention is related to CD123 specific multi-chain Chimeric Antigen Receptor as any of the above embodiment, wherein said second or third transmembrane polypeptide comprises a co-stimulatory domain from the cytoplasmic domain of a costimulatory molecule selected from CD28 and/ 4-1BB.

Advantageously, the present invention provides a CD123 specific multi-chain Chimeric Antigen Receptor as above, wherein said co-stimulatory domain is from 4-1BB and comprises a polypeptide of SEQ ID NO.6.

The present invention also provides a CD123 specific multi-chain Chimeric Antigen Receptor as above, wherein said co-stimulatory domain is from CD28 and comprises a polypeptide sequence of SEQ ID NO.7.

The present invention provides a polynucleotide comprising a nucleic acid sequence encoding a CD123 specific multi-chain Chimeric Antigen Receptor according to any one of the above embodiments.

The present invention provides a polynucleotide comprising a nucleic acid sequence encoding a CD123 specific multi-chain Chimeric Antigen Receptor comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or encoding a CD123 specific multi-chain Chimeric Antigen Receptor comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

The present invention provides a vector comprising a polynucleotide as above, preferably a vector encoding a CD123 specific multi-chain Chimeric Antigen Receptor comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or encoding a CD123 specific multi-chain Chimeric Antigen Receptor comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

A method of engineering an immune cell endowing a CD123 specific multi-chain Chimeric Antigen Receptor according to any one of the above embodiments is part of the present invention, said method of engineering an immune cell is comprising the following steps:
(a) Providing an immune cell;
(b) Expressing at the surface of said cells at least one CD123 multi-chain Chimeric Antigen Receptor according to any one of the above embodiments.

In one embodiment, the present invention provides method of engineering an immune cell endowing a CD123 specific multi-chain Chimeric Antigen Receptor according to any one of the above embodiments comprising:
(a) Providing an immune cell;
(b) Introducing into said cell at least one polynucleotide encoding polypeptides composing a CD123 multi-chain Chimeric Antigen Receptor according to any one of the above; preferably encoding the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or encoding a CD123 specific multi-chain Chimeric Antigen Receptor comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.
(c) Expressing said polynucleotides into said cell.

In a preferred embodiment, said method of engineering an immune cell is comprising:
(a) Providing an immune cell;
(b) Expressing at the surface of said cell a population of CD123 multi-chain

Chimeric Antigen Receptors according to any one of the above embodiments each one comprising different extracellular ligand-binding domains.

In a preferred embodiment, the method of engineering an immune cell is further comprising:
(a) Providing an immune cell;
(b) Introducing into said cell at least one polynucleotide encoding polypeptides composing a population of CD123 multi-chain Chimeric Antigen Receptors according to any one of the above embodiments each one comprising different extracellular ligand binding domains.
(c) Expressing said polynucleotides into said cell.

Thus, the present invention provides an isolated immune cell obtainable from the method according to any one of the above embodiments, preferably an isolated immune cell expressing a CD123 multi-chain Chimeric Antigen Receptors comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or an isolated immune cell expressing a CD123 specific multi-chain Chimeric Antigen Receptor comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

### Cells

The present invention provides an isolated cell said isolated cell is selected from the group consisting of inflammatory T-lymphocytes, cytotoxic T-lymphocytes, regulatory T-lymphocytes or helper T-lymphocytes, preferably said isolated cell further comprises at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6.

The present invention provides an isolated cell said isolated cell is selected from the group consisting of inflammatory T-lymphocytes, cytotoxic T-lymphocytes, regulatory T-lymphocytes or helper T-lymphocytes, said isolated cell further comprises at least one anti-CD123 multi-chain (CAR) comprising comprises at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

In a preferred embodiment, said isolated cell provided in the present invention is an isolated immune T cell and said isolated immune T cell expresses at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6.

In another preferred embodiment, said isolated immune cell is an isolated immune T cell and said isolated immune T cell expresses at least one anti-CD123 multi-chain (CAR) comprising comprises at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

In one embodiment, said isolated immune cell is further engineered and is an engineered primary isolated immune cell comprising at least one anti-CD123 multi-chain (CAR) of the invention.

In a preferred embodiment, said engineered primary isolated immune cell comprises at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6.

In another preferred embodiment, said engineered primary isolated immune cell comprises at least one anti-CD123 multi-chain (CAR) comprising comprises at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

### Pharmaceutical Composition

In one aspect, the present invention provides a pharmaceutical composition as described above.

The present invention provides pharmaceutical composition comprising at least one pharmaceutically acceptable vehicle and at least one primary cell endowed with at least one anti-CD123 multi-chain (CAR).

In one embodiment said pharmaceutical composition comprises at least one pharmaceutically acceptable vehicle and at least one primary cell endowed with at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6.

In one embodiment said pharmaceutical composition comprises at least one pharmaceutically acceptable vehicle and at least one primary cell endowed with at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

### Medicament

The present invention provides an isolated immune cell according to above embodiments for its use as a medicament, preferably an isolated immune T cell endowed with a CD123 mc CAR of the invention for its use as a medicament.

Advantageously, said isolated immune T cell for use as a medicament comprises at least one CD123 mc CAR according to above embodiments. More advantageously, the present application provides an isolated immune T cell for use as a medicament comprising at least one a CD123 mc CAR, said CD123 mcCAR comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or an isolated immune T cell for use as a medicament comprising at least one a CD123 mc CAR, said CD123 mcCAR comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

Even more advantageously, the present application provides an isolated immune T cell comprising at least one CD123 mcCAR comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 for its use as a medicament or an isolated immune T cell comprising at least one CD123 mcCAR comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7 for its use as a medicament.

### Therapeutic Indications

T cells comprising an anti CD123 multi-chain CAR of the invention are provided as a treatment in patients diagnosed with a pre-malignant or malignant cancer condition characterized by CD123-expressing cells, especially by an overabundance of CD123-expressing cells. Such conditions are found in hematologic cancers, such as leukemia, lymphoid malignancies or malignant lymphoproliferative disorders, in carcinoma, blastoma, and sarcoma, and melanomas.

The present application provides T cells comprising an anti CD123 multi-chain CAR comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 for the treatment of carcinoma, blastoma, and sarcoma, and certain leukemia or lymphoid malignancies, benign and malignant tumors, and malignancies e.g., sarcomas, carcinomas, and melanomas.

The present application thus provides T cells comprising an anti CD123 multi-chain CAR comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7 for the treatment of carcinoma, blastoma, and sarcoma, and certain leukemia or lymphoid malignancies, benign and malignant tumors, and malignancies e.g., sarcomas, carcinomas, and melanomas.

In a preferred embodiment, the present application provides T cells comprising an anti CD123 multi-chain CAR comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 for the treatment of leukemia or lymphoid malignancies.

In a preferred embodiment, the present application provides T cells comprising an anti CD123 multi-chain CAR comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7 for the treatment of leukemia or lymphoid malignancies.

The present invention provides T cells comprising an anti CD123 multi-chain CAR comprising either SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7 for use in the treatment of acute myelogenous leukemia (AML), chronic myelogenous leukemia, melodysplastic syndrome, acute lymphoid leukemia, chronic lymphoid leukemia, and myelodysplastic syndrome.

In one embodiment, T cells comprising an anti CD123 multi-chain CAR comprising either SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7 are provided for the treatment of acute myelogenous leukemia (AML).

In one embodiment the present application provides an isolated immune T cells endowed with at least one CD123 mc CAR, comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 for its use as a medicament to prevent or treat refractory/relapse AML
or an isolated immune T cells endowed with at least one with at least one CD123 mc CAR comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7 for its use as a medicament to prevent or treat refractory /relapse AML.

The present invention provides an isolated immune NK cell endowed with at least one CD123 mc CAR, comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 for its use as a medicament to prevent or treat AML
or an isolated immune NK cells endowed with at least one with at least one CD123 mc CAR comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7 for its use as a medicament to prevent or treat AML.

The present invention provides an isolated inflammatory T-lymphocyte endowed with at least one CD123 mc CAR, comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 for its use as a medicament to prevent or treat AML
or an isolated inflammatory-T lymphocytes endowed with at least one with at least one CD123 mc CAR comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7 for its use as a medicament to prevent or treat AML.

The present invention provides an cytotoxic T-lymphocyte endowed with at least one CD123 mc CAR, comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 for its use as a medicament to prevent or treat AML or
an isolated cytotoxic T lymphocyte endowed with at least one with at least one CD123 mc CAR comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7 for its use as a medicament to prevent or treat AML.

The present invention provides an regulatory T-lymphocyte endowed with at least one CD123 mc CAR, comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 for its use as a medicament to prevent or treat AML or
an isolated regulatory T lymphocytes endowed with at least one with at least one CD123 mc CAR comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7 for its use as a medicament to prevent or treat AML.

The present invention provides a helper T-lymphocyte endowed with at least one CD123 mc CAR, comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 for its use as a medicament to prevent or treat AML
or an isolated helper T lymphocyte endowed with at least one with at least one CD123 mc CAR comprising the following peptide sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7 for its use as a medicament to prevent or treat AML.

In another aspect the present invention provides a pharmaceutical composition as above for use as a medicament.

In a preferred aspect the present invention provides a pharmaceutical composition for use as a medicament for the prevention or treatment of a pathological condition such as cancer, in particular a cancer of hematopoietic cells, more particularly AML or MM.

In a preferred embodiment said pathological condition is refractory/relapse AML.

The pharmaceutical composition of the invention for use as a medicament to prevent or treat AML comprises engineered primary immune cells, preferably primary immune T cells, comprising an anti CD123 multi-chain CAR comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7, at least one pharmaceutically acceptable vehicle.

Preferably, the present invention provides a method for treating a patient in need thereof comprising:
a) Providing an isolated immune T cell obtainable by a method according to any one of the above embodiments;
b) Administrating said T-cells to said patient,
wherein said patients is suffering from a cancer selected from AML, MM, ALL CLL, preferably AML, more preferably refractory /relapse AML.

The present invention provides a method for treating a patient as above wherein said immune cells are recovered from donors.

The present invention provides a method for treating a patient as above wherein said immune cells are recovered from a patient, preferably from the patient itself, the patient to be treated by said method.

### Multi-chain Chimeric Antigen Receptor (CAR)

The present invention relates to a multi-chain chimeric antigen receptor (CAR) particularly adapted to immune cells used in immunotherapy.

The multi-chain CAR according to the invention generally comprises at least:
one transmembrane polypeptide comprising at least one extracellular ligand-binding domain and;
one transmembrane polypeptide comprising at least one signal-transducing domain;
   such that said polypeptides assemble together to form a multi-chain Chimeric Antigen Receptor.

The term “extracellular ligand-binding domain” as used herein is defined as an oligo- or polypeptide that is capable of binding a ligand. Preferably, the domain will be capable of interacting with a cell surface molecule. More preferably, said domain will be capable of interacting with a CD123 cell surface molecule.

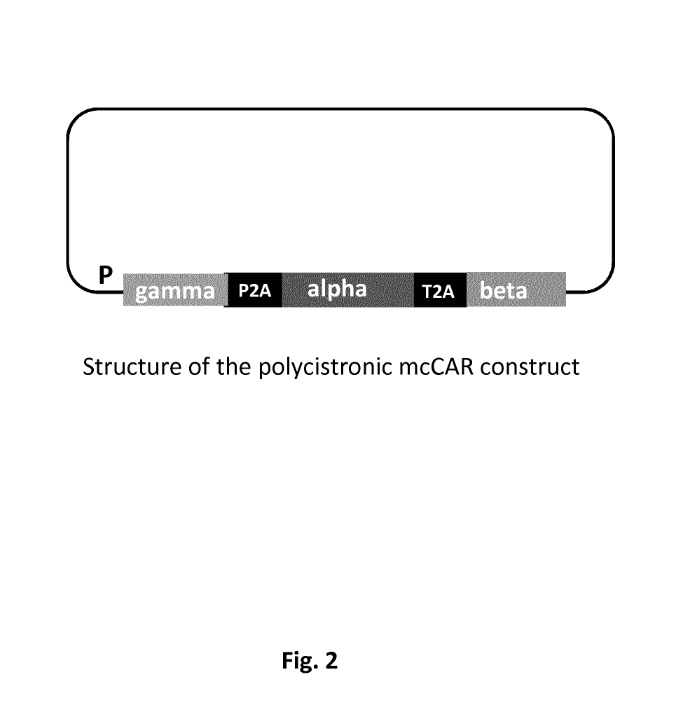

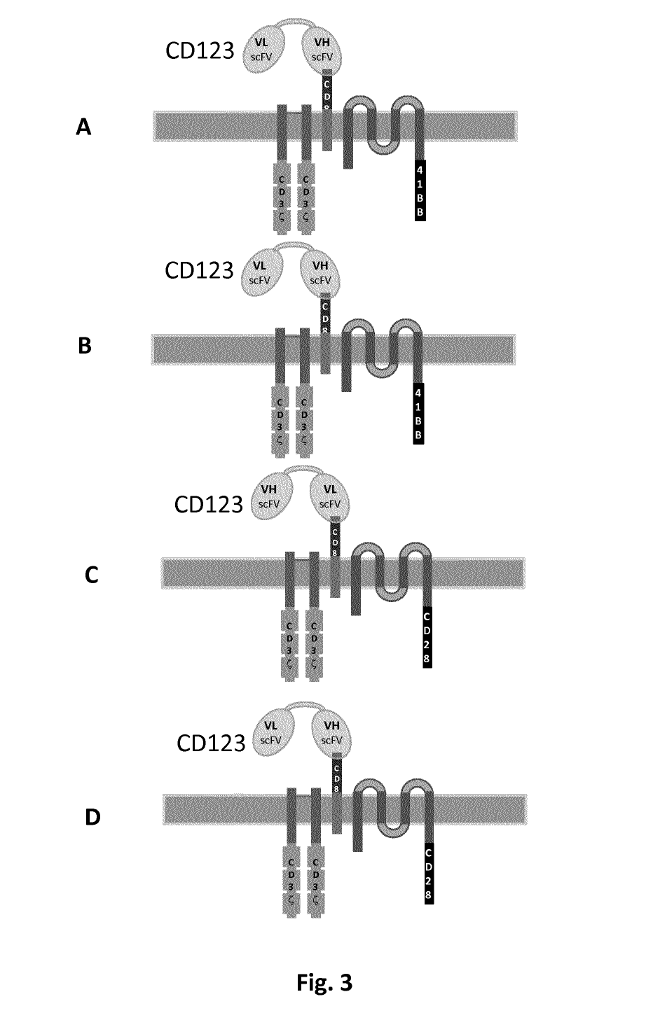

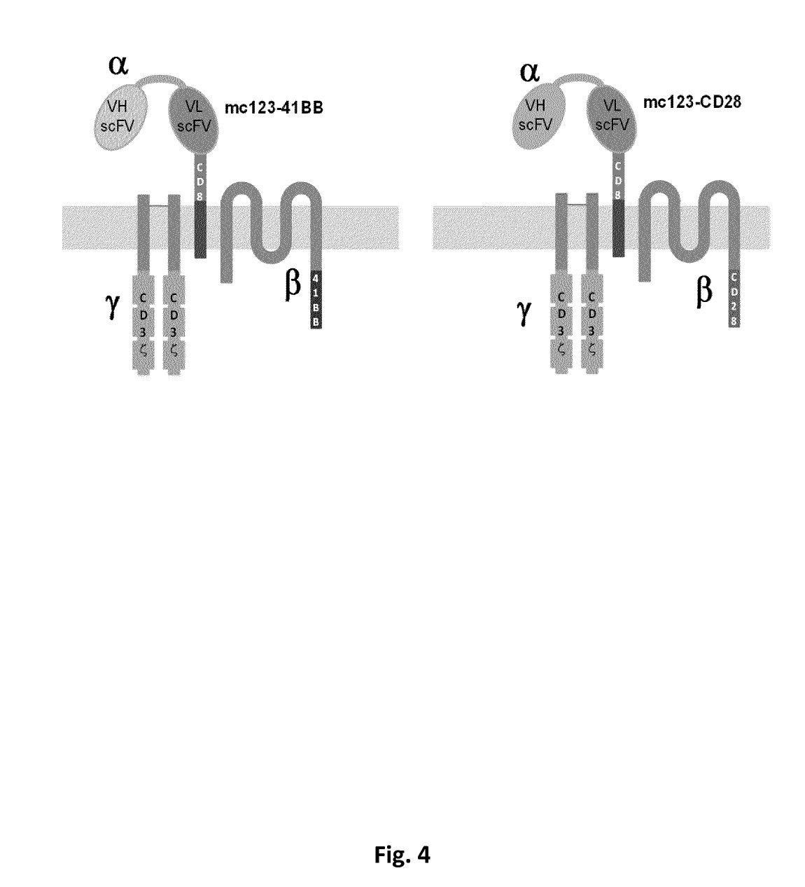

The present invention provides: an anti-CD123 multi-chain chimeric antigen receptor (CAR) (123 mcCAR anti-CD123 mc) having a structure as illustrated in FIG. 2, FIG. 3, or FIG. 4, and according to claim 1, 2 and/or 3 said structure comprising an extra cellular ligand binding-domain VH and VL from a monoclonal anti-CD123 antibody comprising the following CDR sequences:
GFTFTDYY (SEQ ID NO. 26), RSKADGYTT (SEQ ID NO. 27), ARDAAYYSYYSPEGAMDY (SEQ ID NO. 28), and QNVDSA (SEQ ID NO. 29), SAS (SEQ ID NO. 30), QQYYSTPWT (SEQ ID NO. 31), and a hinge between VH and VL (alpha chain),
said structure further comprising:
a cytoplasmic transmembrane domain including a CD3 zeta signaling domain (gamma chain) and
a co-stimulatory transmembrane domain from 4-1BB or CD28 (beta chain).

In a preferred embodiment the present invention provides an anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6.

In another the present invention provides an anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

In a more preferred embodiment said anti-CD123 CARs are constructed with these sequences and correspond to the constructions illustrated in FIG. 4.

In a preferred embodiment, said extracellular ligand-binding domain is a single chain antibody fragment (scFv) comprising the light (V_{L}) and the heavy (V_{H}) variable fragment of a target antigen specific monoclonal antibody specific to CD123 joined by a flexible linker. In a preferred embodiment, said scFv is an anti-CD123 scFV, preferably provided in Table 5 as SEQ ID NO.13 to 24, and more preferably as SEQ ID NO.21 and 22 Binding domain specific to CD123 other than scFv can also be used for predefined targeting of lymphocytes, such as camelid or shark (VNAR) single-domain antibody fragments or receptor ligands like a vascular endothelial growth factor polypeptide, an integrin-binding peptide, heregulin or an IL-13 mutein, antibody binding domains, antibody hypervariable loops or CDRs as non-limiting examples.

In a preferred embodiment said first transmembrane polypeptide further comprises a stalk region between said extracellular ligand-binding domain and said transmembrane domain. The term “stalk region” used herein generally means any oligo- or polypeptide that functions to link the transmembrane domain to the extracellular ligand-binding domain. In particular, stalk region are used to provide more flexibility and accessibility for the extracellular ligand-binding domain. A stalk region may comprise up to 300 amino acids, preferably 10 to 100 amino acids and most preferably 25 to 50 amino acids. Stalk region may be derived from all or part of naturally occurring molecules, such as from all or part of the extracellular region of CD8, CD4 or CD28, or from all or part of an antibody constant region. Alternatively the stalk region may be a synthetic sequence that corresponds to a naturally occurring stalk sequence, or may be an entirely synthetic stalk sequence. In a preferred embodiment said stalk region is a part of human CD8 alpha chain (e.g. NP_001139345.1) (SEQ ID NO: 2).

Thus, the expression of multi-chain CAR in immune cells results in modified cells that selectively and eliminate defined targets, including but not limited to malignant cells carrying a respective tumor-associated surface antigen or virus infected cells carrying a virus-specific surface antigen, or target cells carrying a lineage-specific or tissue-specific surface antigen.

Downregulation or mutation of target antigens is commonly observed in cancer cells, creating antigen-loss escape variants. Thus, to offset tumor escape and render immune cell more specific to target, the multi-chain CAR can comprise several extracellular ligand-binding domains, to simultaneously bind different elements in target thereby augmenting immune cell activation and function. In one embodiment, the extracellular ligand-binding domains can be placed in tandem on the same transmembrane polypeptide, and optionally can be separated by a linker. In another embodiment, said different extracellular ligand-binding domains can be placed on different transmembrane polypeptides composing the multi-chain CAR. In another embodiment, the present invention relates to a population of multi-chain CARs comprising each one different extracellular ligand binding domains. In a particular, the present invention relates to a method of engineering immune cells comprising providing an immune cell and expressing at the surface of said cell a population of multi-chain CAR each one comprising different extracellular ligand binding domains. In another particular embodiment, the present invention relates to a method of engineering an immune cell comprising providing an immune cell and introducing into said cell polynucleotides encoding polypeptides composing a population of multi-chain CAR each one comprising different extracellular ligand binding domains. In a particular embodiment the method of engineering an immune cell comprises expressing at the surface of the cell at least a part of FcϵRI beta and/or gamma chain fused to a signal-transducing domain and several part of FcϵRI alpha chains fused to different extracellular ligand binding domains. In a more particular embodiment, said method comprises introducing into said cell at least one polynucleotide which encodes a part of FcϵRI beta and/or gamma chain fused to a signal-transducing domain and several FcϵRI alpha chains fused to different extracellular ligand binding domains. By population of multi-chain CARs, it is meant at least two, three, four, five, six or more multi-chain CARs each one comprising different extracellular ligand binding domains. The different extracellular ligand binding domains according to the present invention can preferably simultaneously bind different elements in target thereby augmenting immune cell activation and function.

Cells

The present invention also relates to an isolated immune cell which comprises a population of multi-chain CARs each one comprising different extracellular ligand binding domains.

The signal transducing domain or intracellular signaling domain of the multi-chain CAR of the invention is responsible for intracellular signaling following the binding of extracellular ligand binding domain to the target resulting in the activation of the immune cell and immune response. In other words, the signal transducing domain is responsible for the activation of at least one of the normal effector functions of the immune cell in which the multi-chain CAR is expressed. For example, the effector function of a T cell can be a cytolytic activity or helper activity including the secretion of cytokines. Thus, the term “signal transducing domain” refers to the portion of a protein which transduces the effector signal function signal and directs the cell to perform a specialized function.

Preferred examples of signal transducing domain for use in multi-chain CAR can be the cytoplasmic sequences of the Fc receptor or T cell receptor and co-receptors that act in concert to initiate signal transduction following antigen receptor engagement, as well as any derivate or variant of these sequences and any synthetic sequence that as the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non limiting examples those derived from TCRzeta, FcRgamma, FcRbeta, FcRepsilon, CD3gamma, CD3delta, CD3epsilon, CD5, CD22, CD79a, CD79b and CD66d. In a preferred embodiment, the signaling transducing domain of the multi-chain CAR can comprise the CD3zeta signaling domain, or the intracytoplasmic domain of the FcϵRI beta or gamma chains.

In particular embodiment the signal transduction domain of the multi-chain CAR of the present invention comprises a co-stimulatory signal molecule. A co-stimulatory molecule is a cell surface molecule other than an antigen receptor or their ligands that is required for an efficient immune response.

“Co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-IBB, OX40, CD30, CD40, PD-1, ICOS, lymphocyte function-associated antigen-1 (LFA-1), CD2, CD7, LTGHT, NKG2C, B7-H3, a ligand that specifically binds with CD83.

A “co-stimulatory molecule” refers to the cognate binding partner on a T-cell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor. Examples of costimulatory molecules include CD27, CD28, CD8, 4-1BB (CD137), 0X40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

In another particular embodiment, said signal transducing domain is a TNFR-associated Factor 2 (TRAF2) binding motifs, intracytoplasmic tail of costimulatory TNFR member family. Cytoplasmic tail of costimulatory TNFR family member contains TRAF2 binding motifs consisting of the major conserved motif (P/S/A)X(Q/E)E) or the minor motif (PXQXXD), wherein X is any amino acid. TRAF proteins are recruited to the intracellular tails of many TNFRs in response to receptor trimerization.

In a preferred embodiment, the signal transduction domain of the multi-chain CAR of the present invention comprises a part of co-stimulatory signal molecule selected from the group consisting of 4-1BB (GenBank: AAA53133.) and CD28 (NP_006130.1).

The distinguishing features of appropriate transmembrane polypeptides comprise the ability to be expressed at the surface of an immune cell, in particular lymphocyte cells or Natural killer (NK) cells, and to interact together for directing cellular response of immune cell against a predefined target cell. The different transmembrane polypeptides of the multi-chain CAR of the present invention comprising an extracellular ligand-biding domain and/or a signal transducing domain interact together to take part in signal transduction following the binding with a target ligand and induce an immune response. The transmembrane domain can be derived either from a natural or from a synthetic source. The transmembrane domain can be derived from any membrane-bound or transmembrane protein. As non limiting examples, the transmembrane polypeptide can be a subunit of the T cell receptor such as α, β, γ or [symbol] , polypeptide constituting CD3 complex, IL2 receptor p55 (α chain), p75 (β chain) or γ chain, subunit chain of Fc receptors, in particular Fcγ receptor III or CD proteins. Alternatively the transmembrane domain can be synthetic and can comprise predominantly hydrophobic residues such as leucine and valine.

The term “derived from” means a polypeptide having an amino acid sequence which is equivalent to that an Fcϵ receptor which include one or more amino acid modification(s) of the sequence of the Fcϵ receptor. Such amino acid modification(s) may include amino acid substitution(s), deletion(s), addition(s) or a combination of any of those modifications, and may alter the biological activity of the Fc binding region relative to that of an Fc receptor. On the other hand, Fc binding regions derived from a particular Fc receptor may include one or more amino acid modification(s) which do not substantially alter the biological activity of the Fc binding region relative to that of an Fc receptor. Amino acid modification(s) of this kind will typically comprise conservative amino acid substitution(s).

In a particular embodiment, the multi-chain CAR comprises a transmembrane polypeptide derived from a FcϵRI chain. In more particular embodiment FcϵRI chain is a FcϵRI α chain, in which the extracellular domain is replaced by an extracellular ligand-binding domain, preferably by a scFV directed against CD123.

In more particular embodiment, said multi-chain CAR can comprise a part of FcϵRI alpha chain and a part of FcϵRI beta chain or variant thereof such that said FcϵRI chains spontaneously dimerize together to form a dimeric Chimeric Antigen Receptor. In another embodiment, the multi-chain Chimeric Antigen can comprise a part of FcϵRI alpha chain and a part of a FcϵRI gamma chain or variant thereof such that said FcϵRI chains spontaneously trimerize together to form a trimeric Chimeric Antigen Receptor, and in another embodiment the multi-chain Chimeric Antigen Receptor can comprise a part of FcϵRI alpha chain, a part of FcϵRI beta chain and a part of FcϵRI gamma chain or variants thereof such that said FcϵRI chains spontaneously tetramerize together to form a tetrameric Chimeric Antigen Receptor.

As non-limiting example, different versions (architectures) of multi-chain CAR are illustrated in FIG. 3. In a preferred embodiment, two versions (architectures) of multi-chain CAR are illustrated in FIG. 4. In a more preferred embodiment, the multi-chain CARs of the present invention comprises a polypeptide comprising amino acid sequences as set forth in Table 6. In another preferred embodiment the multi-chain CAR comprise a polypeptide with amino acid sequence that has at least 70%, preferably at least 80%, more preferably at least 90%, 95% 97% or 99% sequence identity with such amino amino acid sequences or with the polynucleotide sequence encoding one two or three of the polypeptides constitutive of the multi-chain polypeptide structure.

In a more preferred embodiment, the invention provided is a cell endowed with a multi-chain anti-CD123 CAR of the invention comprising a polypeptide with amino acid sequence comprising the following CDR sequences:

GFTFTDYY (SEQ ID NO. 26), RSKADGYTT (SEQ ID NO. 27), ARDAAYYSYYSPEGAMDY (SEQ ID NO. 28), and QNVDSA (SEQ ID NO. 29), SAS (SEQ ID NO. 30), QQYYSTPWT (SEQ ID NO. 31), a hinge between VH and VL (alpha chain),

said multi-chain anti-CD123 CAR further comprising:
a cytoplasmic transmembrane domain including a CD3 zeta signaling domain (gamma chain) and
a co-stimulatory transmembrane domain from 4-1BB or CD28 (beta chain).

In a more preferred embodiment, the invention provided is a cell endowed with a multi-chain anti-CD123 CAR of the invention comprising a polypeptide with amino acid sequence comprising the following sequences: SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 or the following sequences SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

### Polynucleotides, Vectors

The present invention also relates to polynucleotides, vectors encoding the above described multi-chain CAR according to the invention. The present invention provides polynucleotides, including DNA and RNA molecules that encode the transmembrane polypeptides disclosed herein that can be included in the multi-chain CAR. In particular, the invention relates to a polynucleotide comprising a nucleic acid sequence encoding at least one transmembrane polypeptide composing the multi-chain CAR as described above. More particularly the invention relates to a polynucleotide comprising two or more nucleic acid sequences encoding transmembrane polypeptides composing the multi-chain CAR as described above.

The polynucleotide may consist in an expression cassette or expression vector (e.g. a plasmid for introduction into a bacterial host cell, or a viral vector such as a baculovirus vector for transfection of an insect host cell, or a plasmid or viral vector such as a lentivirus for transfection of a mammalian host cell).

In a particular embodiment, the different nucleic acid sequences can be included in one polynucleotide or vector which comprises a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see Donnelly et al., J. of General Virology 82: 1013-1025 (2001); Donnelly et al., J. of Gen. Virology 78: 13-21 (1997); Doronina et al., Mol. And. Cell. Biology 28(13): 4227-4239 (2008); Atkins et al., RNA 13: 803-810 (2007)). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA. As non-limiting example, in the present invention, 2A peptides have been used to express into the cell the different polypeptides of the multi-chain CAR.

To direct, transmembrane polypeptide such as FcϵR into the secretory pathway of a host cell, a secretory signal sequence (also known as a leader sequence, prepro sequence or pre sequence) is provided in polynucleotide sequence or vector sequence. The secretory signal sequence may be that of FcϵR, or may be derived from another secreted protein (e.g., t-PA) or synthesized de novo. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences may be positioned elsewhere in the nucleic acid sequence of interest (see, e.g., Welch et al., U.S. Pat. No. 5,037,743; Holland et al., U.S. Pat. No. 5,143,830). In a preferred embodiment the signal peptide comprises the residues 1 to 25 of the FcϵRI alpha chain (NP_001992.1) and has the amino acid sequence SEQ ID NO: 5.

Those skilled in the art will recognize that, in view of the degeneracy of the genetic code, considerable sequence variation is possible among these polynucleotide molecules. Preferably, the nucleic acid sequences of the present invention are codon-optimized for expression in mammalian cells, preferably for expression in human cells. Codon-optimization refers to the exchange in a sequence of interest of codons that are generally rare in highly expressed genes of a given species by codons that are generally frequent in highly expressed genes of such species, such codons encoding the amino acids as the codons that are being exchanged.

The present invention also relates to polynucleotides, vectors encoding the anti-CD123 multi-chain CAR of the invention.

In a preferred embodiment said polynucleotides, vectors encoding the anti-CD123 multi-chain CAR of the invention encodes an anti-CD123 multi-chain CAR comprising the following CDR sequences:
GFTFTDYY (SEQ ID NO. 26), RSKADGYTT (SEQ ID NO. 27), ARDAAYYSYYSPEGAMDY (SEQ ID NO. 28), and QNVDSA (SEQ ID NO. 29), SAS (SEQ ID NO. 30), QQYYSTPWT (SEQ ID NO. 31), a hinge between VH and VL (alpha chain),
   a cytoplasmic transmembrane domain including a CD3 zeta signaling domain (gamma chain) and
   a co-stimulatory transmembrane domain from 4-1BB or CD28 (beta chain).

The present invention provides polynucleotides, vectors encoding an anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6.

In another embodiment, the present invention provides polynucleotides, vectors encoding an anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

### Methods of Engineering an Immune Cell

In encompassed particular embodiment, the invention relates to a method of preparing immune cells for immunotherapy comprising introducing into said immune cells the polypeptides composing said multi-chain CAR and expanding said cells. In particular embodiment, the invention relates to a method of engineering an immune cell comprising providing a cell and expressing at the surface of said cell at least one multi-chain CAR as described above. In particular embodiment, the method comprises transforming the cell with at least one polynucleotide encoding polypeptides composing at least one multi-chain CAR as described above, and expressing said polynucleotides into said cell.

In another embodiment, the present invention relates to a method of preparing cells for immunotherapy comprising introducing into said cells the different polypeptides composing said multi-chain CAR and expanding said cells. In a preferred embodiment, said polynucleotides are included in lentiviral vectors in view of being stably expressed in the cells.

The invention relates to a method of preparing primary immune cells for immunotherapy comprising introducing into said primary immune cells the polypeptides composing said anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6.

In another embodiment, the present invention provides a method of preparing primary immune cells for immunotherapy comprising introducing into said immune cells the polypeptides composing said
anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

### Delivery Methods

The different methods described above involve introducing multi-chain CAR, pTalpha or functional variants thereof, rare cutting endonuclease, TALE-nuclease, CAR optionally with DNA-end processing enzyme or exogenous nucleic acid into a cell.

As non-limiting example, said multi-chain CAR can be introduced as transgenes encoded by one or as different plasmidic vectors. Different transgenes can be included in one vector which comprises a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see Donnelly et al., J. of General Virology 82: 1013-1025 (2001); Donnelly et al., J. of Gen. Virology 78: 13-21 (1997); Doronina et al., Mol. And. Cell. Biology 28(13): 4227-4239 (2008); Atkins et al., RNA 13: 803-810 (2007)). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA. As non-limiting example, in the present invention, 2A peptides have been used to express into the cell the rare-cutting endonuclease and a DNA end-processing enzyme or the different polypeptides of the multi-chain CAR.

Said plasmid vector can also contain a selection marker which provides for identification and/or selection of cells which received said vector.

Polypeptides may be synthesized in situ in the cell as a result of the introduction of polynucleotides encoding said polypeptides into the cell. Alternatively, said polypeptides could be produced outside the cell and then introduced thereto. Methods for introducing a polynucleotide construct into animal cells are known in the art and including as non-limiting examples stable transformation methods wherein the polynucleotide construct is integrated into the genome of the cell, transient transformation methods wherein the polynucleotide construct is not integrated into the genome of the cell and virus mediated methods. Said polynucleotides may be introduced into a cell by for example, recombinant viral vectors (e.g. retroviruses, adenoviruses), liposome and the like. For example, transient transformation methods include for example microinjection, electroporation or particle bombardment. Said polynucleotides may be included in vectors, more particularly plasmids or virus, in view of being expressed in cells.

Electroporation

In particular embodiment of the invention, polynucleotides encoding polypeptides according to the present invention can be mRNA which is introduced directly into the cells, for example by electroporation. The inventors determined the optimal condition for mRNA electroporation in T-cell.

The inventor used the cytoPulse technology which allows, by the use of pulsed electric fields, to transiently permeabilize living cells for delivery of material into the cells. The technology, based on the use of PulseAgile (Cellectis property) electroporation waveforms grants the precise control of pulse duration, intensity as well as the interval between pulses (U.S. Pat. No. 6,010,613 and International PCT application W02004083379). All these parameters can be modified in order to reach the best conditions for high transfection efficiency with minimal mortality. Basically, the first high electric field pulses allow pore formation, while subsequent lower electric field pulses allow moving the polynucleotide into the cell. In one aspect of the present invention, the inventor describe the steps that led to achievement of >95% transfection efficiency of mRNA in T cells, and the use of the electroporation protocol to transiently express different kind of proteins in T cells. In particular the invention relates to a method of transforming T cell comprising contacting said T cell with RNA and applying to T cell an agile pulse sequence consisting of:
(a) one electrical pulse with a voltage range from 2250 to 3000 V per centimeter, a pulse width of 0.1 ms and a pulse interval of 0.2 to 10 ms between the electrical pulses of step (a) and (b);
(b) one electrical pulse with a voltage range from 2250 to 3000 V with a pulse width of 100 ms and a pulse interval of 100 ms between the electrical pulse of step (b) and the first electrical pulse of step (c); and
(c) 4 electrical pulses with a voltage of 325 V with a pulse width of 0.2 ms and a pulse interval of 2 ms between each of 4 electrical pulses.

In particular embodiment, the method of transforming T cell comprising contacting said T cell with RNA and applying to T cell an agile pulse sequence consisting of:
(a) one electrical pulse with a voltage of 2250, 2300, 2350, 2400, 2450, 2500, 2550, 2400, 2450, 2500, 2600, 2700, 2800, 2900 or 3000V per centimeter, a pulse width of 0.1 ms and a pulse interval of 0.2, 0.5, 1, 2, 3, 4, 5, 6, 7, 8, 9 or 10 ms between the electrical pulses of step (a) and (b);
(b) one electrical pulse with a voltage range from 2250, of 2250, 2300, 2350, 2400, 2450, 2500, 2550, 2400, 2450, 2500, 2600, 2700, 2800, 2900 or 3000V with a pulse width of 100 ms and a pulse interval of 100 ms between the electrical pulse of step (b) and the first electrical pulse of step (c); and
(c) 4 electrical pulses with a voltage of 325 V with a pulse width of 0.2 ms and a pulse interval of 2 ms between each of 4 electrical pulses.

Any values included in the value range described above are disclosed in the present application. Electroporation medium can be any suitable medium known in the art. Preferably, the electroporation medium has conductivity in a range spanning 0.01 to 1.0 milliSiemens.

In particular embodiments, as non-limiting examples, said RNA encodes a rare-cutting endonuclease, one monomer of the rare-cutting endonuclease such as Half-TALE-nuclease, a Chimeric Antigen Receptor, at least one component of the multi-chain chimeric antigen receptor, a pTalpha or functional variant thereof, an exogenous nucleic acid, one additional catalytic domain.

### Engineered T-Cells

The present invention also relates to isolated cells or cell lines susceptible to be obtained by said method to engineer cells. In particular said isolated cell comprises at least one multi-chain CAR as described above. In another embodiment, said isolated cell comprises a population of multi-chain CARs each one comprising different extracellular ligand binding domains. In particular, said isolated cell comprises exogenous polynucleotide sequences encoding polypeptides composing at least one multi-chain CAR.

In the scope of the present invention is also encompassed an isolated immune cell, preferably a T-cell obtained according to any one of the methods previously described.

Said immune cell refers to a cell of hematopoietic origin functionally involved in the initiation and/or execution of innate and/or adaptative immune response. Said immune cell according to the present invention can be derived from a stem cell. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells.

In a preferred embodiment, said isolated cell is an isolated stem CD34+ cell, said isolated stem CD34+ cell comprises at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6.

In another preferred embodiment, said isolated cell is an isolated stem CD34+ cell, said isolated stem CD34+ cell comprises at least one anti-CD123 multi-chain (CAR) comprising comprises at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

Said isolated cell can also be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes or helper T-lymphocytes. In another embodiment, said cell can be derived from the group consisting of CD4+ T-lymphocytes and CD8+ T-lymphocytes.

Prior to expansion and genetic modification of the cells of the invention, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art, may be used. In another embodiment, said cell can be derived from a healthy donor, from a patient diagnosed with cancer or from a patient diagnosed with an infection. In another embodiment, said cell is part of a mixed population of cells which present different phenotypic characteristics. In the scope of the present invention is also encompassed a cell line obtained from a transformed T- cell according to the method previously described. Modified cells resistant to an immunosuppressive treatment and susceptible to be obtained by the previous method are encompassed in the scope of the present invention. As mentioned previously, such cells can be also genetically engineered to inactivate one or several genes selected, for instance, from the group consisting of CD52, GR, TCR alpha, TCR beta, HLA gene, immune check point genes such as PD1 and CTLA-4, or can express a pTalpha transgene.

In another embodiment, TCR is rendered not functional in the cells according to the invention by inactivating TCR alpha gene and/or TCR beta gene(s). The above strategies are used more particularly to avoid GvHD. In a particular aspect of the present invention is a method to obtain modified cells derived from an individual, wherein said cells can proliferate independently of the Major Histocompatibility Complex signaling pathway. Said method comprises the following steps:
(a) Recovering cells from said individual;
(b) Genetically modifying said cells ex-vivo by inactivating TCR alpha and/or TCR beta genes;
(c) Cultivating genetically modified T-cells in vitro in appropriate conditions to amplify said cells.

The present invention provides primary engineered T cell comprising at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6.

The present invention provides primary engineered T cell comprising at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7.

Modified cells, which can proliferate independently of the Major Histocompatibility Complex signaling pathway, susceptible to be obtained by this method are encompassed in the scope of the present invention. Said modified cells can be used in a particular aspect of the invention for treating patients in need thereof against Host versus Graft (HvG) rejection and Graft versus Host Disease (GvHD); therefore in the scope of the present invention is a method of treating patients in need thereof against Host versus Graft (HvG) rejection and Graft versus Host Disease (GvHD) comprising treating said patient by administering to said patient an effective amount of modified cells comprising inactivated TCR alpha and/or TCR beta genes.

In a more preferred embodiment, said method comprises:
(a) Providing a T-cell, preferably from a cell culture or from a blood sample;
(b) Transforming said T cell with nucleic acid encoding a rare-cutting endonuclease able to selectively inactivate by DNA cleavage, preferably by double-strand break at least one gene encoding a component of the T-cell receptor (TCR);
(c) Expressing said rare-cutting endonucleases into said T-cells;
(d) Sorting the transformed T-cells, which do not express TCR on their cell surface;
(e) Expanding said cells.

In another embodiment, said rare-cutting endonuclease can be a meganuclease, a Zinc finger nuclease or a TALE-nuclease. In a preferred embodiment, said rare-cutting endonuclease is a TALE-nuclease. Preferred methods and relevant TALE-nucleases have been described in WO2013176915.

The present invention provides primary engineered T cell comprising at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6 inducing from 50% to 100% less Host versus Graft (HvG) rejection than primary non engineered T cell.

The present invention provides primary engineered T cell comprising at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7, inducing from 50% to 100% less Host versus Graft (HvG) rejection than primary non engineered T cell.

### Anti-CD123 Immune Cells Made Resistant to Chemotherapy

According to a preferred embodiment of the invention, the immune cells endowed with an anti CD123 multi-chain CAR are engineered to be resistant to chemotherapy drugs, in particular to purine nucleotide analogues (PNAs), making them suitable for cancer treatments in order to combine adoptive immunotherapy and chemotherapy. Purine nucleotide analogues enter chemotherapy compositions for many cancer treatments, especially leukemia. It is particularly used as a standard of care in AML. The most widely used PNAs are clofarabine, fludarabine and cytarabine, alone or in combination. PNAs are metabolized by enzymes having deoxycytidine kinase (dCK) activity [EC 2.7.1.74] into mono, -di and tri-phosphate PNA. Their tri-phosphate forms and particularly clorofarabine triphosphate compete with ATP for DNA synthesis, acts as pro-apotptotic agent and are potent inhibitors of ribonucleotide reductase (RNR), which is involved in trinucleotide production.

The present invention thus includes a method of producing ex-vivo immune cells, preferably T-cells, which are resistant to a purine analogue drug and that can target CD123 positive malignant cells. Said method comprises one or several of the following steps of:
(a) Providing an immune cell from a patient (autologous treatment) or from a donor;
(b) transfecting said immune cell with a nucleic acid sequence encoding a rare-cutting endonuclease specifically targeting a gene expressing an enzyme having deoxycytidine kinase activity (dcK—EC 2.7.1.74), in particular the human deoxycytidine kinase gene (NCBI Gene ID: 1633).
(c) expressing said endonuclease into said immune cells to obtain targeted inactivation of said dck gene;
(d) Expanding the engineered immune cells obtained in step c), optionally in the presence of said purine analogue drug; and
(e) Introducing into said immune cell an anti-CD123 multi chain CAR as previously described.

The present inventors have successfully created anti-CD123 T-cells resistant to purine nucleotide analogues, more particularly clorofarabine and/or fludarabine, by mediating the inactivation of dcK gene expression into said cells particularly by using TAL-nucleases. Transfection of the T-cells using mRNA encoding specific TAL-nuclease directed against cdk genes, preferably by using electroporation as described in W02013176915, induced a significant resistance to the drugs, while maintaining T-cells cytotoxic activity towards CD123 bearing cells.

The present application thus provides with anti-CD123 T-cells, which expression of deoxycytidine kinase has been repressed or inactivated for the treatment of leukemia.

The present invention provides primary engineered T cell comprising at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.6, in which expression of deoxycytidine kinase has been repressed or inactivated for the treatment of leukemia, preferably AML

The present invention provides primary engineered T cell comprising at least one anti-CD123 multi-chain (CAR) comprising SEQ ID NO.8, SEQ ID NO.9, SEQ ID NO.10, SEQ ID NO.11, SEQ ID NO.1, SEQ ID NO.2, SEQ ID NO.21, SEQ ID NO.3, SEQ ID NO.22, SEQ ID NO.4, SEQ ID NO.12, SEQ ID NO.5, and SEQ ID NO.7, in which expression of deoxycytidine kinase has been repressed or inactivated for the treatment of leukemia, preferably AML.

### Activation and Expansion of T Cells

Whether prior to or after genetic modification of the T cells, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo.

Generally, the T cells of the invention are expanded by contact with an agent that stimulates a CD3 TCR complex and a co-stimulatory molecule on the surface of the T cells to create an activation signal for the T-cell.

For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. For example, the agents providing each signal may be in solution or coupled to a surface. As those of ordinary skill in the art can readily appreciate, the ratio of particles to cells may depend on particle size relative to the target cell. In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, 1L-4, 1L-7, GM-CSF, -10, −2, 1L-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth; for example, an appropriate temperature (e.g., 37° C.) and atmosphere (e.g., air plus 5% C02). T cells that have been exposed to varied stimulation times may exhibit different characteristics

In another particular embodiment, said cells can be expanded by co-culturing with tissue or cells. Said cells can also be expanded in vivo, for example in the subject's blood after administrating said cell into the subject.

### Therapeutic Applications

In another embodiment, isolated cell obtained by the different methods or cell line derived from said isolated cell as previously described can be used as a medicament. In another embodiment, said medicament can be used for treating cancer or infections in a patient diagnosed with a pathology linked to CD123 positive cells. In another embodiment, said isolated cell according to the invention or cell line derived from said isolated cell can be used in the manufacture of a medicament for treatment of a cancer, especially AML.

In another aspect, the present invention relies on methods for treating patients in need thereof, said method comprising at least one of the following steps:
(a) providing an immune-cell obtainable by any one of the methods previously described;
(b) Administrating said transformed immune cells to said patient,

On one embodiment, said T cells of the invention can undergo robust in vivo T cell expansion and can persist for an extended amount of time.

Said treatment can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

The invention is particularly suited for allogenic immunotherapy, insofar as it enables the transformation of T-cells, typically obtained from donors, into non-alloreactive cells. This may be done under standard protocols and reproduced as many times as needed. The resulted modified T cells may be pooled and administrated to one or several patients, being made available as an “off the shelf” therapeutic product.

Cells that can be used with the disclosed methods are described in the previous section. Said treatment can be used to treat patients diagnosed with cancer, viral infection, autoimmune disorders or Graft versus Host Disease (GvHD). Cancers that may be treated include tumors that are not vascularized, or not yet substantially vascularized, as well as vascularized tumors. The cancers may comprise nonsolid tumors (such as hematological tumors, for example, leukemias and lymphomas) or may comprise solid tumors. Types of cancers to be treated with the multi-chain CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Said treatment can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

Cells that can be used with the disclosed methods are described in the previous section. Said preventive or therapeutic treatment can be used to treat patients diagnosed wherein a pre-malignant or malignant cancer condition characterized by CD123-expressing cells, especially by an overabundance of CD123-expressing cells. Such conditions are found in hematologic cancers, such as leukemia or malignant lymphoproliferative disorders.

It can be a treatment in combination with one or more therapies against cancer selected from the group of antibodies therapy, chemotherapy, cytokines therapy, dendritic cell therapy, gene therapy, hormone therapy, laser light therapy and radiation therapy.

According to a preferred embodiment of the invention, said treatment can be administrated into patients undergoing an immunosuppressive treatment. Indeed, the present invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the patient. The administration of the cells or population of cells according to the present invention may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, said effective amount of cells or composition comprising those cells are administrated parenterally. Said administration can be an intravenous administration. Said administration can be directly done by injection within a tumor.

In certain embodiments of the present invention, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizimab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p7056 kinase that is important for growth factor induced signaling (rapamycin) (Liu et al., Cell 66:807-815, 1 1; Henderson et al., Immun. 73:316-321, 1991; Bierer et al., Citrr. Opin. mm n. 5:763-773, 93). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery. Said modified cells obtained by any one of the methods described here can be used in a particular aspect of the invention for treating patients in need thereof against Host versus Graft (HvG) rejection and Graft versus Host Disease (GvHD); therefore in the scope of the present invention is a method of treating patients in need thereof against Host versus Graft (HvG) rejection and Graft versus Host Disease (GvHD) comprising treating said patient by administering to said patient an effective amount of modified (engineered) cells comprising inactivated TCR alpha and/or TCR beta genes.

In the present application a patient or a subject means non-human primates or humans.

A donor means a healthy individual or an individual suffering from a disease.

The term “relapsed” refers to a situation where a subject who has had a remission of cancer after therapy has a return of cancer cells.

The term “refractory or resistant” refers to a circumstance where a subject or a mammal, even after intensive treatment, has residual cancer cells in his body.

The term “drug resistance” refers to the condition when a disease does not respond to the treatment of a drug or drugs. Drug resistance can be either intrinsic (or primary resistance), which means the disease has never been responsive to the drug or drugs, or it can be acquired, which means the disease ceases responding to a drug or drugs that the disease had previously responded to (secondary resistance). In certain embodiments, drug resistance is intrinsic. In certain embodiments, the drug resistance is acquired.

The term “hematologic malignancy” or “hematologic cancer” refers to a cancer of the body's blood- bone marrow and/or lymphatic tissue. Examples of hematological malignancies include, for instance, myelodysplasia, leukemia, lymphomas, such as cutaneous Lymphomas, non-Hodgkin's lymphoma, Hodgkin's disease (also called Hodgkin's lymphoma), and myeloma, such as acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), and multiple myeloma (MM).

The term “leukemia” refers to malignant neoplasms of the blood-forming tissues, including, but not limited to, chronic lymphocytic leukemia or chronic lymphoid leukemia, chronic myelocytic leukemia, or chronic myelogenous leukemia, acute lymphoblastic leukemia, acute myeloid leukemia or acute myelogenous leukemia (AML) and acute myeloblastic leukemia.

AML or AML subtypes that may be treated using the anti CD123 multi-chain CAR-expressing cells of the present invention may be in particular, acute myeloblastic leukemia, minimally differentiated acute myeloblastic leukemia, acute myeloblastic leukemia without maturation, acute myeloblastic leukemia with granulocytic maturation, promyelocytic or acute promyelocytic leukemia (APL), acute myelomonocytic leukemia, myelomonocytic together with bone marrow eosinophilia, acute monoblastic leukemia (M5a) or acute monocytic leukemia (M5b), acute erythroid leukemias, including erythroleukemia (M6a) and very rare pure erythroid leukemia (M6b), acute megakaryoblastic leukemia, acute basophilic leukemia, acute panmyelosis with myelofibrosis, whether involving CD123-positive malignat cells.

Subtypes of AML also include, hairy cell leukemia, Philadelphia chromosome-positive acute lymphoblastic leukemia.

AML or AML subtypes that may be treated using the anti CD123 multi-chain CAR-expressing cells of the present invention may be AML with specific genetic abnormalities. Classification is based on the ability of karyotype to predict response to induction therapy, relapse risk, survival.

Accordingly, AML that may be treated using the anti CD123 multi-chain CAR—expressing cells of the present invention may be AML with a translocation between chromosomes 8 and 21, AML with a translocation or inversion in chromosome 16, AML with a translocation between chromosomes 9 and 11, APL (M3) with a translocation between chromosomes 15 and 17, AML with a translocation between chromosomes 6 and 9, AML with a translocation or inversion in chromosome 3, AML (megakaryoblastic) with a translocation between chromosomes 1 and 22 .

The present invention is particularly useful for the treatment of AML associated with these particular cytogenetic markers.

The present invention also provides an anti CD123 multi-chain CAR -expressing cells for the treatment of patients with specific cytogenetic subsets of AML, such as patients with t(15;17)(q22;q21) identified using all-trans retinoic acid (ATRA)16-19 and for the treatment of patients with t(8;21)(q22;q22) or inv(16)(p13q22)/t(16;16)(p13;q22) identified using repetitive doses of high-dose cytarabine.

Preferably, the present invention provides an anti CD123 multi-chain CAR -expressing cells for the treatment of AML suffering patients with aberrations, such as -5/del(5q), -7, abnormalities of 3q, or a complex karyotype, who have been shown to have inferior complete remission rates and survival.

In specific embodiments “comprising” means “consisting in”.

### General Methods

All methods disclosed in document PCT/EP2015/055848 are incorporated herein by references

Primary T-Cell Cultures

T cells were purified from Buffy coat samples provided by EFS (Etablissement Français du Sang, Paris, France) using Ficoll gradient density medium (Ficoll Paque PLUS/GE Healthcare Life Sciences). The PBMC layer was recovered and T cells were purified using a commercially available T-cell enrichment kit (Stem Cell Technologies). Purified T cells were activated in X-Vivo™-15 medium (Lonza) supplemented with 20 ng/mL Human IL-2 (Miltenyi Biotech), 5% Human Serum (Sera Laboratories), and Dynabeads Human T activator CD3/CD28 at a bead:cell ratio 1:1 (Life Technologies). After activation cells were grown and maintained in X-Vivo™-15 medium (Lonza) supplemented with 20 ng/mL Human IL-2 (Miltenyi Biotec) and 5% Human Serum (Sera Laboratories).

Models of AML and Clorofarabine, Fludarabine or Cytarabine Resistant AML

Originally, MOLM13 cell line has been established from the peripheral blood of a 20-year-old man with acute myeloid leukemia AML FAB M5a at relapse in 1995 after initial myelodysplastic syndromes (MDS, refractory anemia with excess of blasts, RAEB). To establish the MOLM13-Luc cell line and dck Knock out MOLM13-Luc cell line (clorofarabine, fludarabine or cytarabine resistant MOLM13-Luc cell line), MOLM13 cells (DSMZ ACC 554) were transfected with a nucleic acid sequence encoding a rare-cutting endonuclease specifically targeting a gene expressing an enzyme having deoxycytidine kinase activity (dcK—EC 2.7.1.74), namely the human deoxycytidine kinase gene (NCBI Gene ID: 1633), and with a lentivirus encoding the GFP and the firefly luciferase (amsbio LVP438-PBS).
The GFP-positive cells have been selected with Neomycin (ref 10131-027, Gibco, Life Technologies, Saint-Aubin, France). Resistance to clorofarabine, fludarabine or cytarabine of cdk KO MOLM13-Luc cells was tested in the presence of clorofarabine, fludarabine or cytarabine.

T-Cell Transduction and CAR Detection

Transduction of T-cells with recombinant lentiviral vectors along the expression of scar or mcCAR was carried out three days after T-cell purification/activation. Lentiviral vectors were produced by Vectalys SA (Toulouse, France) by transfection of genomic and helper plasmids in HEK-293 cells. Transductions were carried out at a multiplicity of infection of 5, using 10⁶ cells per transduction. CAR detection at the surface of T-cells was done using a recombinant protein consisting on the fusion of the extracellular domain of the human CD123 protein together with a murine IgG1 Fc fragment (produced by LakePharma). Binding of this protein to the CAR molecule was detected with a PE-conjugated secondary antibody (Jackson Immunoresearch) targeting the mouse Fc portion of the protein, and analyzed by flow cytometry.

Degranulation Assay (CD107a Mobilization)

T-cells were incubated in 96-well plates (40,000 cells/well), together with an equal amount of cells expressing or not the CD123 protein. Co-cultures were maintained in a final volume of 100 μl of X-Vivo™-15 medium (Lonza) for 6 hours at 37° C. with 5% CO₂. CD107a staining was done during cell stimulation, by the addition of a fluorescent anti-CD107a antibody (APC conjugated, from Miltenyi Biotec) at the beginning of the co-culture, together with 1 μg/ml of anti-CD49d (BD Pharmingen), 1 μg/ml of anti-CD28 (Miltenyi Biotec), and 1× Monensin solution (eBioscience). After the 6 h incubation period, cells were stained with a fixable viability dye (eFluor 780, from eBioscience) and fluorochrome-conjugated anti-CD8 (PE conjugated Miltenyi Biotec) and analyzed by flow cytometry.

The degranulation activity was determined as the % of CD8+/CD107a+ cells, and by determining the mean fluorescence intensity signal (MFI) for CD107a staining among CD8+ cells. Degranulation assays were carried out 8-10 days after T-cell transduction with mcCAR or scCAR.

Cytotoxicity Assay

T-cells were incubated in 96-well plates (100,000 cells/well), together with 10,000 target cells (expressing various levels of CD123) and 10,000 control (CD123neg) cells in the same well. Target and control cells were labelled with fluorescent intracellular dyes (CFSE or Cell Trace Violet, from Life Technologies) before co-culturing them with CAR+ T-cells (mcCAR+ T-cells or scCAR+ T-cells). The co-cultures were incubated for 4 hours at 37° C. with 5% CO₂. After this incubation period, cells were labelled with a fixable viability dye (eFluor 780, from eBioscience) and analyzed by flow cytometry. Viability of each cellular population (target cells or CD123neg control cells) was determined and the % of specific cell lysis was calculated. Cytotoxicity assays were carried out 48 h after mRNA transfection.

Anti-Tumor Mouse Model
Animal housing and experimental procedures were carried out by Oncodesign (Dijon, France; http://www.oncodesign.com/), according to the French and European Regulations and NRC Guide for the Care and Use of Laboratory Animals.
Immunodefficient female NOG (NOG) mice (NOD.Cg-PrkdcscidII2rgtm1Sug/JicTac) mice (NOD stands for non-obese diabetic), 6-8 weeks old, were obtained from Taconic (Ry, Danemark).
In one arm of the experiment, mice received clorofarabine or fludarabine.
   Mice were intravenously (iv) injected with MOLM13-Luciferase cells or with clorofarabine resistant MOLM13-Luciferase cells as an AML and an clorofarabine resistant AML mouse model, respectively.

Mice were then iv injected (7 days after injection of the tumor cell line) with different doses of mcCAR+ T-cells or scCAR+ T-cells (from 10⁴ to 5×10⁶), or with T-cells that were not transduced with any CAR lentiviral vector.

Bioluminescent signals were determined the day before T-cell injection (D-1) and at D7 and 14 after T-cell injection, in order to follow tumoral progression on the different animals.

The results show a dose dependent alteration of tumoral progression in mice treated with mcCAR+ T-cells, as illustrated FIG. 4.

Sequence of the anti-CD123 scCAR used in the present application was as follows:

(SEQ. ID NO. 25)
MALPVTALLLPLALLLHAARPEVKLVESGGGLVQPGGSLSLSCAASGFTF
TDYYMSWVRQPPGKALEWLALIRSKADGYTTEYSASVKGRFTLSRDDSQS
ILYLQMNALRPEDSATYYCARDAAYYSYYSPEGAMDYWGQGTSVTVSSGG
GGSGGGGSGGGGSMADYKDIVMTQSHKFMSTSVGDRVNITCKASQNVDSA
VAWYQQKPGQSPKALIYSASYRYSGVPDRFTGRGSGTDFTLTISSVQAED
LAVYYCQQYYSTPWTFGGGTKLEIKRTTTPAPRPPTPAPTIASQPLSLRP
EACRPAAGGAVHTRGLDFACDIYIWAPLAGTCGVLLLSLVITLYCKRGRK
KLLYIFKQPFMRPVQTTQEEDGCSCRFPEEEEGGCELRVKFSRSADAPAY
QQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR

### Clinical Essay

Examples of Conditions:

Patients with newly diagnosed AML.

Patients with relapsed or refractory AML or patients with AML who are not eligible for intensive treatment.

### Example of CD123 Specific Multi-Chain CARs

A. Design of Multi-Chain CARs (FIG. 2, FIG. 3, FIG. 4)

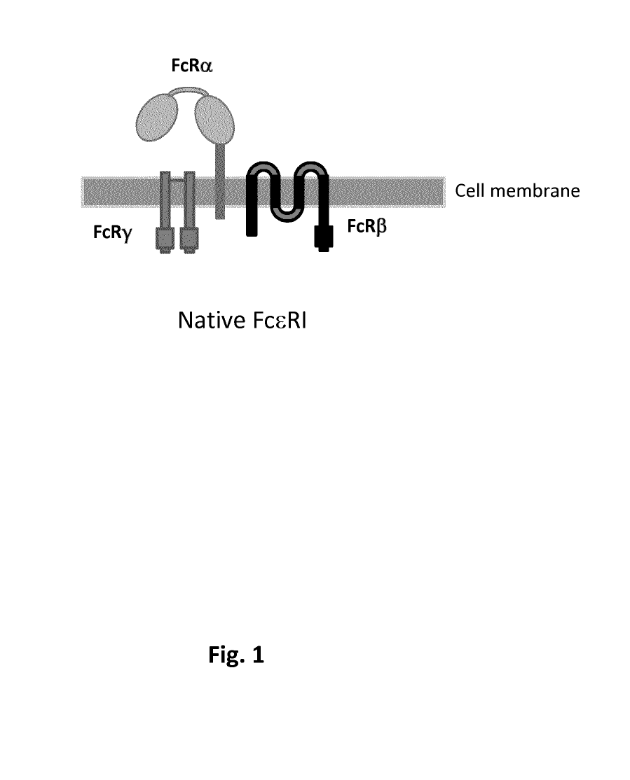

Ten multi-chain CARs targeting the CD123 antigen were designed based on the high affinity receptor for IgE (FcϵRI). The FcϵRI (FIG. 1) expressed on mast cells and basophiles triggers allergic reactions. It is a tetrameric complex composed of a single α subunit, a single β subunit and two disulfide-linked γ subunits. The α subunit contains the IgE-binding domain. The β and γ subunits contain ITAMs that mediate signal transduction. In every multi-chain CAR, the extracellular domain of the FcRα chain was deleted and replaced by the respective scFv referred to μln Table 5 respectively and the CD8α hinge (SEQ ID NO: 2) and the ITAM of the FcRβ chain and/or the FcRγ chain was deleted. The resulting constructions had the structure detailed in table 6.

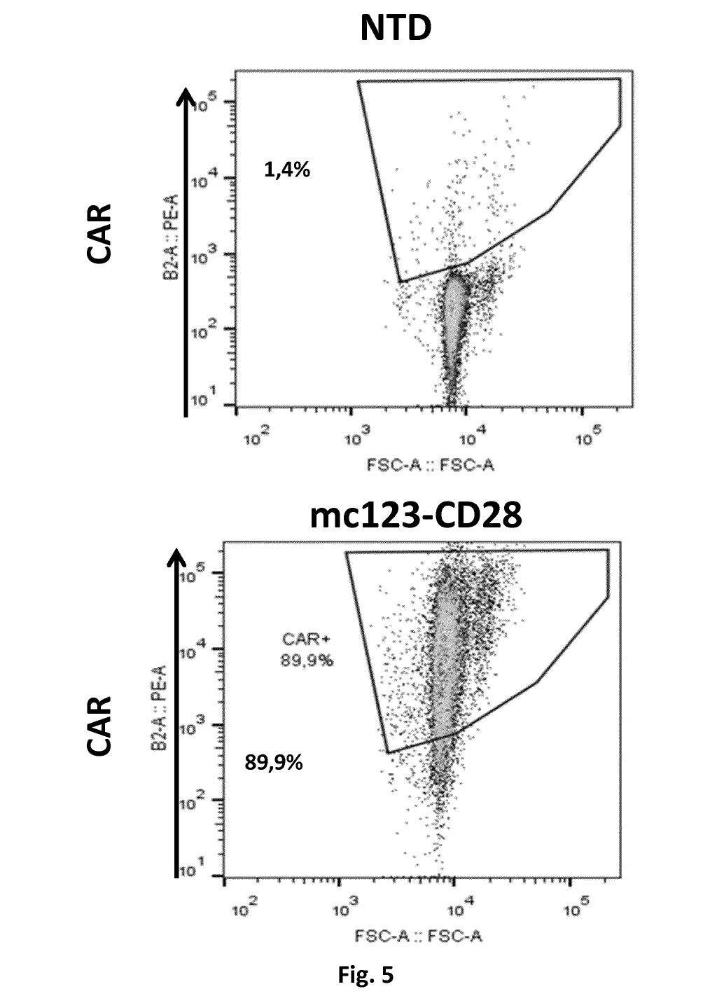

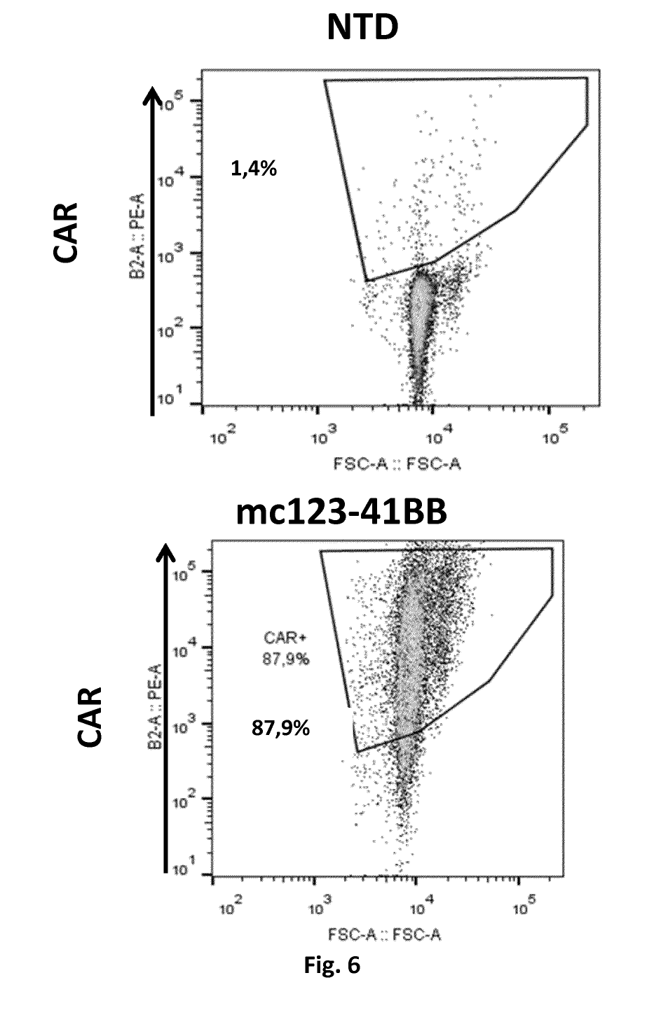

B. Transiently expression in T cells (FIG. 5, FIG. 6)

### Multi-Chain CARs Can Be Expressed in Human T Cells After Electroporation of Polycistronic MRNA.

T cells were electroporated with capped and polyadenylated polycistronic mRNA that were produced using the mMESSAGE mMACHINE kit and linearized plasmids as template. The plasmids used as template contained the T7 RNA polymerase promoter followed by a polycistronic DNA sequence encoding the different CAR variants.

The electroporation of the polycistronic mRNAs into the human T cells was done using the CytoLVT-S device (Cellectis), according to the following protocol: 5×10⁶ T cells preactivated several days (3-5) with anti CD3/CD28 coated beads and IL2 were resuspended in cytoporation buffer T, and electroporated in 0.4cm cuvettes with 45 μg of mRNA using the PBMC3 program Table 14.

24 hours post electroporation, human T cells engineered using polycistronic mRNAs encoding the multi-chain CARs were labeled with a fixable viability dye eFluor-780 and a PE-conjugated goat anti mouse IgG F(ab')2 fragment specific, and analyzed by flow cytometry.

The live T cells engineered using polycistronic mRNAs expressed the multi-chain CARs on their surface.

The results in FIG. 5 and FIG. 6 show expression levels of each of the mcCAR assessed 8 days after transduction at MOI 5. CAR detection was done using a recombinant fusion protein containing the extracellular domain of the human CD123 protein, fused to a mouse IgG1 derived Fc fragment. The CAR/CD123-Fc complex was detected with a PE-conjugated anti-Fc antibody and analyzed by flow cytometry. NTD stands for Non Transduced cells. FIG. 5 shows 89.9% cells expressing mc123-CD28 and FIG. 6 shows 87.9% cells expressing mc123-41BB as compared to control.

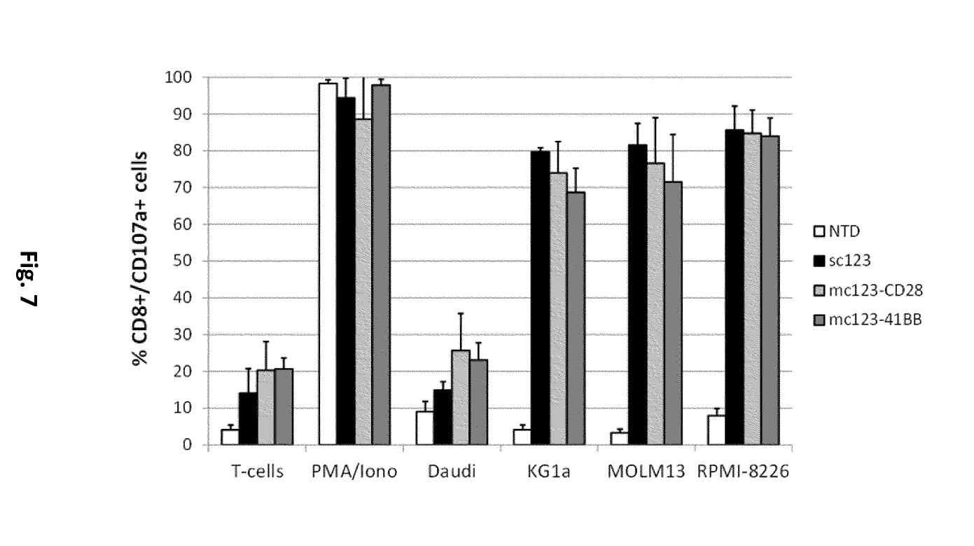

C. The Human T Cells Transiently Expressing the Multi-Chain CARs Degranulate Following Coculture with Target Cells (FIG. 7).

24 hours post electroporation, human T cells engineered using polycistronic mRNAs encoding the multi-chain CARs or encoding a single chain CAR prepared as described in PCT/EP2015/055848. Cells were then co-cultured with target (Daudi), KG1, MOLM13 or RPM18226 or control (K562) cells for 6 hours, (expressing different levels of CD123 (KG1a<MOLM13<RPM18226).

The CD8+ T cells were then analyzed by flow cytometry to detect the expression of the degranulation marker CD107a at their surface. The data indicate that the human CD8+ T cells expressing the CD123 multi-chain CARs degranulate in coculture with CD123 expressing target cells but not in coculture with control cells.

The degranulation activity of T-cells cultured alone, in the same conditions that the co-cultures, is also shown FIG. 7; as the well as the positive control (cells activated with PMA/lonomycin). The degranulation activity was determined by flow cytometry, by measuring the % of CD107a+ cells (among CD8+ cells). The experiments were done in at least three independent donors.

D. Secretion of Cytokines in Human T Cells Transiently Expressing the Multi-Chain CARs Following Coculture with Target Cells

24 hours post electroporation, human T cells engineered using polycistronic mRNAs encoding the multi-chain CARs were co-cultured with target (Daudi) or control (K562) cells for 24 hours. The supernatants were then harvested and analyzed using the TH1/TH2 cytokine cytometric bead array kit to quantify the cytokines produced by the T cells. The assay indicated that the human T cells expressing the multi-chain CARs produce IFNγ, IL8 and IL5 in coculture with CD123 expressing target cells but not in coculture with control cells. Interestingly, anti-CD123 mc CAR-T cells induced a lower level of cytokine release than anti-CD123 scCAR-T cells when used at the same dose, less secondary effect are observed and T cells are better tolerated, thus deemed to be less toxic.

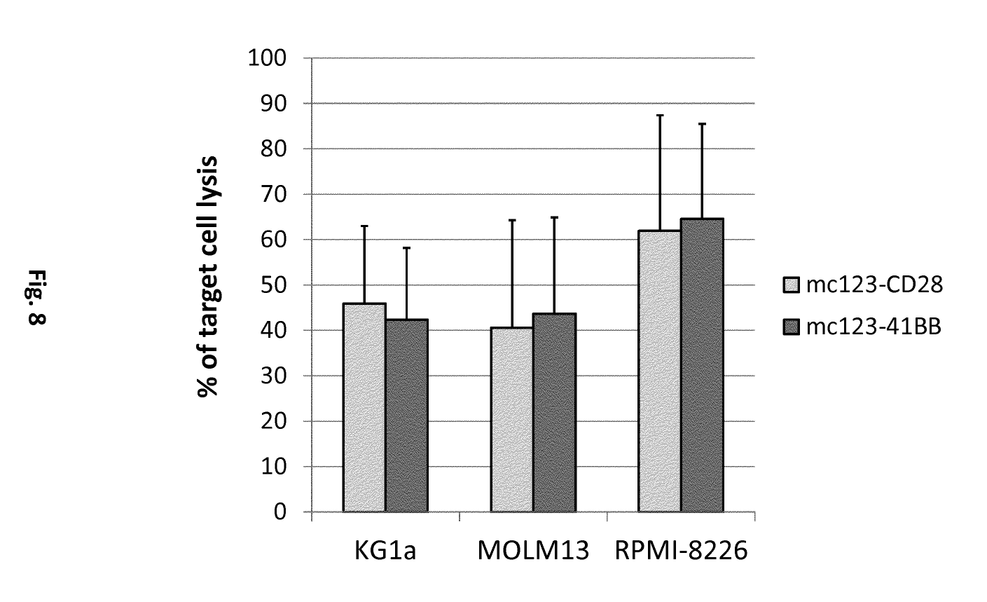

E. The Human T Cells Transiently Expressing the Multi-Chain CARs Lyse Target Cells (FIG. 8)

24 hours post electroporation, human T cells engineered using polycistronic mRNAs encoding the multi-chain CARs were co-cultured with target (Daudi) or control (K562) cells for 4 hours. The target cells were then analyzed by flow cytometry to analyze their viability. indicating that the different cells expressing the CD123 multi-chain CARs lyse the CD123 expressing target cells but not the control cells.

The results in FIG. 8 show the specific cytolytic activity of CAR-T cells. T-cells were co-cultured with Daudi+KG1a, Daudi+MOLM13, or Daudi+RPMI-8226 cells for 4 hours. Cellular viability for each of the cell lines was determined at the end of the co-cultures and a specific cell lysis percentage was calculated for each condition.

The results in FIG. 8 show that both mcCAR targeting CD123 have comparable cytolytic activity against CD123+ target cells.

F—The Human T Cells Transiently Expressing the Multi-Chain CARs Has Anti-Tumor Activity

The two CARs were also used to carry out antitumor in vivo experiments in a tumor established mouse model. Immunodefficient mice were intravenously (iv) injected with (cdk wt or cdk KO) MOLM13-Luciferase cells 7 days before iv injection of non-transduced human T-cells, or with different doses of anti-CD123 mcCAR+ T-cells (from 10⁴ to 5×10⁶) (TCR KO drug resistant engineered cells). The results show a dose dependent anti-tumor activity of anti-CD123 mcCAR+ T-cells in mice.

### G—Clinical Data Related to GVHD

Data obtained indicates that anti-tumor activity of TCR KO CD123 CART cells is similar to that of TCR KO CD123 CART cells. A dramatic improvement in the tolerance (about 50% to 80% reduction of GVHD as compared to TCR expressing cells) of KO CD123 CAR T cells is measured in individuals treated with these cells as compared to cells.

1) A CD123 specific multi-chain Chimeric Antigen Receptor (mc CAR) comprising:
A transmembrane polypeptide from the alpha chain of high-affinity IgE receptor (FcϵRI) fused to an extracellular CD123 ligand binding domain.

2) A CD123 specific multi-chain Chimeric Antigen Receptor (mc CAR) according to claim 1 further comprising:
A second transmembrane polypeptide from the gamma or beta chain of FcϵRI fused to a signal transducing domain;

3) A CD123 specific multi-chain Chimeric Antigen Receptor (mc CAR) according to claim 2, further comprising:
A third transmembrane polypeptide from the gamma or beta chain of FcϵRI comprising a co-stimulatory domain.

4) A CD123 specific multi-chain Chimeric Antigen Receptor according to claim 1, wherein said CD123 ligand binding domain fused to said alpha chain of FcϵRI is a single-chain variable fragment (scFv) comprising heavy (V_{H}) and light (V_{L}) chains conferring specificity to CD123.

5) A CD123 specific multi-chain Chimeric Antigen Receptor of claim 4, wherein said V_{H} comprises a polypeptide sequence displaying at least 90% identity to one selected from SEQ ID NO. 13, 15, 17, 19, 21 and 23.

6) A CD123 specific multi-chain Chimeric Antigen Receptor of claim 1, wherein said V_{L} comprises a polypeptide displaying at least 90% identity to one selected from SEQ ID NO. 14, 16, 18, 20, 22 and 24.

7) A CD123 specific multi-chain Chimeric Antigen Receptor of claim 1, wherein said alpha chain of FcϵRI is fused to said extracellular ligand-binding domain by a hinge from CD8a, IgG1 or FcRIIIα proteins.

8) A CD123 specific multi-chain Chimeric Antigen Receptor of claim 1, wherein said hinge comprises a polypeptide sequence displaying at least 90% identity to SEQ ID NO.2.

9) A CD123 specific multi-chain Chimeric Antigen Receptor according to claim 2, wherein said signal transducing domain fused to the gamma or beta chain of FcϵRI is from the TCR zeta chain, the FCϵRβ chain, the FcϵRIγ chain, or includes an immunoreceptor tyrosine-based activation motif (ITAM).

10) A CD123 specific multi-chain Chimeric Antigen Receptor according to claim 9, wherein said signal transducing domain is from CD3zeta.

11) A CD123 specific multi-chain Chimeric Antigen Receptor according to claim 10, wherein said signal transducing domain comprises a polypeptide sequence displaying at least 90% identity to SEQ ID NO.10.

12) A CD123 specific multi-chain Chimeric Antigen Receptor according to claim 3, wherein said second or third polypeptide comprises a co-stimulatory domain from the cytoplasmic domain of a costimulatory molecule selected from CD27, CD28, 4-1BB, OX40, CD30, CD40, PD-1, ICOS, lymphocyte function-associated antigen-1 (LFA-1), CD2, CD7, CD8, LIGHT, NKG2C, B7-H3, a ligand that specifically binds with CD83, and any combination thereof.

13) A CD123 specific multi-chain Chimeric Antigen Receptor according to claim 12, wherein said co-stimulatory domain is from 4-1BB and comprises a polypeptide sequence displaying at least 90% identity to SEQ ID NO.6.

14) A CD123 specific multi-chain Chimeric Antigen Receptor according to claim 12, wherein said co-stimulatory domain is from CD28 and comprises a polypeptide sequence displaying at least 90% identity to SEQ ID NO.7.

15) A polypeptide encoding a CD123 specific multi-chain Chimeric Antigen Receptor according to claim 1, comprising a polypeptide sequence displaying at least 80% identity to the full amino acid sequence of anti-CD123 7G3, anti-CD123 Old4, anti-CD123 26292, anti-CD123 32716, anti-CD123 Klon43, anti-CD123 12F1 as referred to in Table 6.

16) A polynucleotide comprising a nucleic acid sequence encoding a CD123 specific multi-chain Chimeric Antigen Receptor according to claim 1.

17) A vector comprising a polynucleotide of claim 16.

18) A method of engineering an immune cell comprising:
(a) Providing an immune cell;
(b) Expressing at the surface of said cells at least one multi-chain Chimeric Antigen Receptor according to claim 1.

19) The method of engineering an immune cell of claim 18 comprising:
(a) Providing an immune cell;
(b) Introducing into said cell at least one polynucleotide encoding polypeptides composing at least one multi-chain Chimeric Antigen Receptor;
(c) Expressing said polynucleotides into said cell.

20) The method of engineering an immune cell of claim 18 comprising:
(a) Providing an immune cell;
(b) Expressing at the surface of said cell a population of multi-chain Chimeric Antigen Receptors, each one comprising different extracellular ligand-binding domains.

21) The method of engineering an immune cell of claim 18 comprising:
(a) Providing an immune cell;
(b) Introducing into said cell at least one polynucleotide encoding polypeptides composing a population of multi-chain Chimeric Antigen Receptors, each one comprising different extracellular ligand binding domains.
(c) Expressing said polynucleotides into said cell.

22) An isolated immune cell obtainable from the method according to claim 18.

23) An isolated immune cell comprising at least one multi-chain Chimeric Antigen Receptor according to claim 1.

24) An isolated immune cell according to claim 22 for its use as a medicament.

25) An isolated cell according to any one of claims 22 derived from, NK cells, inflammatory T-lymphocytes, cytotoxic T-lymphocytes, regulatory T-lymphocytes or helper T-lymphocytes.

26) A method for treating a patient in need thereof comprising:
a) Providing a immune cell obtainable by a method according to claim 18;
b) Administrating said T-cells to said patient,

27) The method for treating a patient of claim 26, wherein said immune cells are recovered from donors.

28) The method for treating a patient of claim 26, wherein said immune cells are recovered from patients.


SEQUENCE LISTING (31 sequences)

SEQ ID NO: 1 - protein, 25 aa, homo sapiens
  Fc Receptor for IgE, alpha chain, signal peptide
MAPAMESPTLLCVALLFFAPDGVLA

SEQ ID NO: 2 - protein, 45 aa, homo sapiens
  CD8a-hinge
TTTPAPRPPTPAPTIASQPLSLRPEACRPAAGGAVHTRGLDFACD

SEQ ID NO: 3 - protein, 15 aa, artificial sequence
  linker for VH and VL fragments of the scFv
GGGGSGGGGSGGGGS

SEQ ID NO: 4 - protein, 52 aa, homo sapiens
  Fc Receptor for IgE, alpha chain, transmembrane and intracellular domain
FFIPLLVVILFAVDTGLFISTQQQVTFLLKIKRTRKGFRLLNPHPKPNPKNN

SEQ ID NO: 5 - protein, 215 aa, homo sapiens
  Fc Receptor for IgE, beta chain, without ITAM
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPE

SEQ ID NO: 6 - protein, 42 aa, homo sapiens
  41BB Intracellular domain
KRGRKKLLYIFKQPFMRPVQTTQEEDGCSCRFPEEEEGGCEL

SEQ ID NO: 7 - protein, 41 aa, homo sapiens
  CD28 Intracellular domain
RSKRSRGGHSDYMNMTPRRPGPTRKHYQPYAPPRDFAAYRS

SEQ ID NO: 8 - protein, 18 aa, homo sapiens
  Fc Receptor for IgE, gamma chain, signal peptide
MIPAVVLLLLLLVEQAAA

SEQ ID NO: 9 - protein, 43 aa, homo sapiens
  Fc Receptor for IgE, gamma chain, without ITAM
LGEPQLCYILDAILFLYGIVLTLLYCRLKIQVRKAAITSYEKS

SEQ ID NO: 10 - protein, 112 aa, homo sapiens
  CD3z intracellular domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 22 aa, artificial sequence
  GSG-P2A ribosomal skip peptide
GSGATNFSLLKQAGDVEENPGP

SEQ ID NO: 12 - protein, 21 aa, artificial sequence
  GSG-T2A ribosomal skip peptide
GSGEGRGSLLTCGDVEENPGP

SEQ ID NO: 13 - protein, 141 aa, artificial sequence
  anti-CD123 7G3 heavy chain variable region
MGWSWIFLFLVSGTGGVLSEVQLQQSGPELVKPGASVKMSCKASGYTFTDYYMKWVKQSHGKSLEWIGDIIPSNGATFYNQKFKGKATLTVDRSSSTAYMHLNSLTSEDSAVYYCTRSHLLRASWFAYWGQGTLVTVSAAS

SEQ ID NO: 14 - protein, 134 aa, artificial sequence
  anti-CD123 7G3 light chain variable region
MESQTQVLMSLLFWVSGTCGDFVMTQSPSSLTVTAGEKVTMSCKSSQSLLNSGNQKNYLTWYLQKPGQPPKLLIYWASTRESGVPDRFTGSGSGTDFTLTISSVQAEDLAVYYCQNDYSYPYTFGGGTKLEIKR

SEQ ID NO: 15 - protein, 140 aa, artificial sequence
  anti-CD123 Old4 heavy chain variable region
WTWRFLFVVAAATGVQSQVQLLQSGAEVKKPGSSVKVSCKASGGTFSTYAISWVRQAPGQGLEWMGGIIPIFGIVNYAQKFQGRVTITADESTSTAYMELSSLRSEDTAVYYCARGGGSGPDVLDIWGQGTMVTVSSAST

SEQ ID NO: 16 - protein, 132 aa, artificial sequence
  anti-CD123 Old4 light chain variable region
MDMRVPAQLLGLLLLWLPGARCVIWMTQSPSLLSASTGDRVTISCRMSQGIRSYLAWYQQKPGKAPELLIYAASTLQSGVPSRFSGSGSGTDFTLTISSLQSEDFATYYCQQYYSFPYTFGQGTKLEIKRTV

SEQ ID NO: 17 - protein, 115 aa, artificial sequence
  anti-CD123 26292 heavy chain variable region
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSS

SEQ ID NO: 18 - protein, 107 aa, artificial sequence
  anti-CD123 26292 light chain variable region
DVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIK

SEQ ID NO: 19 - protein, 118 aa, artificial sequence
  anti-CD123 32716 heavy chain variable region
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSS

SEQ ID NO: 20 - protein, 111 aa, artificial sequence
  anti-CD123 32716 light chain variable region
DIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELK

SEQ ID NO: 21 - protein, 127 aa, artificial sequence
  anti-CD123 Klon43 heavy chain variable region
EVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSS

SEQ ID NO: 22 - protein, 113 aa, artificial sequence
  anti-CD123 Klon43 light chain variable region
MADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKR

SEQ ID NO: 23 - protein, 119 aa, artificial sequence
  anti-CD123 12F1 heavy chain variable region
VQLQESGPGLVKPSQSLSLTCSVTDYSITSGYYWNWIRQFPGNKLEWMGYISYDGSNNYNPSLKNRISITRDTSKNQFFLKLSSVTTEDTATYYCSRGEGFYFDSWGQGTTLTVSSARS

SEQ ID NO: 24 - protein, 113 aa, artificial sequence
  anti-CD123 12F1 light chain variable region
DIMMSQSPSSLAVSVGEKFTMTCKSSQSLFFGSTQKNYLAWYQQKPGQSPKLLIYWASTRESGVPDRFTGSGSGTDFTLAISSVMPEDLAVYYCQQYYNYPWTFGGGTKLEIK

SEQ ID NO: 25 - protein, 499 aa, Artificial sequence
  Klo43-3+SP
MALPVTALLLPLALLLHAARPEVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSSGGGGSGGGGSGGGGSMADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 8 aa, Artificial sequence
  CDR1
GFTFTDYY

SEQ ID NO: 27 - protein, 9 aa, Artificial sequence
  CDR2
RSKADGYTT

SEQ ID NO: 28 - protein, 18 aa, Artificial sequence
  CDR3
ARDAAYYSYYSPEGAMDY

SEQ ID NO: 29 - protein, 6 aa, Artificial sequence
  CDR4
QNVDSA

SEQ ID NO: 30 - protein, 3 aa, Artificial sequence
  CDR5
SAS

SEQ ID NO: 31 - protein, 9 aa, Artificial sequence
  CDR6
QQYYSTPWT